(19) United States
(12) Patent Application Publication
(10) Pub. No.: US 2026/0335324 A1
(43) Pub. Date: Oct. 29, 2026

(54) METHODS OF TREATING CHOLANGIOCARCINOMA IN HEPATICALLY IMPAIRED PATIENTS

(71) Applicant: TAIHO PHARMACEUTICAL CO., LTD., Tokyo (JP)

(72) Inventor: VOLKER WACHECK, Princeton, NJ (US)

(21) Appl. No.: 19/648,070

(22) Filed: Apr. 15, 2026

Related U.S. Application Data
(60) Provisional application No. 63/789,569, filed on Apr. 16, 2025.

(51) Int. Cl. A61K 31/519

(52) U.S. Cl. CPC A61K 31/519

(57) ABSTRACT

The present disclosure provides a method of treating cholangiocarcinoma in a subject with hepatic impairment and elevated bilirubin levels, comprising administering to the subject 20 mg of futibatinib once a day. The method may be used to treat previously treated, unresectable, locally advanced or metastatic intrahepatic cholangiocarcinoma harboring fibroblast growth factor receptor 2 (FGFR2) gene fusions or other rearrangements. The pharmacokinetic parameters of the unbound futibatinib in the subject may be increased compared to a subject with normal hepatic function. The subject may be monitored for an increased risk of adverse reactions attributable to higher unbound futibatinib exposure.

### BACKGROUND

Cholangiocarcinoma is a rare and aggressive cancer that originates in the bile ducts. Intrahepatic cholangiocarcinoma specifically develops in bile ducts within the liver. Recent advances in understanding the molecular drivers of this disease have led to the development of targeted therapies for patients with specific genetic alterations.

Fibroblast growth factor receptor 2 (FGFR2) gene fusions and rearrangements are actionable targets in a subset of intrahepatic cholangiocarcinoma cases. These genetic alterations can lead to constitutive activation of FGFR signaling pathways, promoting tumor growth. Consequently, FGFR inhibitors have become an important treatment option for patients with FGFR2-altered cholangiocarcinoma.

Management of cholangiocarcinoma patients is often complicated by concurrent hepatic dysfunction from underlying liver disease, biliary obstruction, or tumor involvement. This presents challenges in drug dosing, as many anticancer agents undergo hepatic metabolism. Patients with impaired liver function may experience altered drug pharmacokinetics, potentially leading to increased toxicity or reduced efficacy.

Hepatic function assessment in cancer patients typically uses classification systems such as the Child-Pugh score or the National Cancer Institute Organ Dysfunction Working Group (NCI-ODWG) criteria. These systems categorize hepatic impairment based on clinical and laboratory parameters, guiding clinicians in making appropriate dosing decisions.

For FGFR inhibitors, understanding how hepatic impairment affects drug exposure and safety is crucial for optimizing dosing strategies while maintaining therapeutic efficacy. However, obtaining comprehensive pharmacokinetic data across different levels of hepatic dysfunction can be challenging, particularly for newer agents.

When treating cholangiocarcinoma with targeted therapies, balancing efficacy and safety becomes more complex in patients with varying degrees of liver function. Robust pharmacokinetic and safety data are needed to guide appropriate dosing, especially for those with moderate to severe hepatic impairment who are often excluded from clinical trials.

Ongoing research aims to refine treatment approaches for cholangiocarcinoma patients by considering both tumor molecular characteristics and overall liver function. This approach maximizes the benefits of targeted therapies while minimizing risks associated with altered drug metabolism in hepatically impaired patients.

Futibatinib, a targeted therapy for intrahepatic cholangiocarcinoma with FGFR2 gene fusions or rearrangements, is primarily metabolized and excreted by the liver. The initial FDA label for futibatinib (revised September 2022) lacked crucial information regarding its pharmacokinetics in patients with moderate or severe hepatic impairment. This absence of data, coupled with the knowledge that futibatinib undergoes extensive hepatic metabolism, raised significant concerns regarding the safety of using futibatinib in this patient population. Without established safety profiles for patients with compromised liver function, clinicians may have been more hesitant to prescribe futibatinib to this patient population compared to an alternative treatment with data on the impact of hepatic impairment on PK (and treatment instructions for patients with hepatic impairment based on this data), since the potential for altered metabolism, clearance, and plasma protein binding in hepatically impaired patients could increase total or unbound drug exposure and increase risk of adverse events. Consequently, clinicians faced a clinical dilemma when considering futibatinib treatment for patients with both intrahepatic cholangiocarcinoma and hepatic impairment, a common comorbidity in this patient group.

The problem of safely administering futibatinib to patients with hepatic impairment is solved by the present invention. This disclosure provides crucial pharmacokinetic data for both total and unbound futibatinib in patients with varying degrees of hepatic impairment that allows for more informed and precise dosing decisions. Specifically, the invention addresses the potential for increased adverse reactions in patients with cirrhosis and elevated total bilirubin levels (>1.5×ULN to 3×ULN) due to higher unbound futibatinib exposure. By providing this detailed pharmacokinetic information, classified by both Child-Pugh and NCI-ODWG criteria, the invention enables healthcare providers to safely administer futibatinib to patients with hepatic impairment, tailoring the treatment approach based on the individual patient's liver function status. This solution significantly expands the treatable patient population for futibatinib, addressing a critical unmet need in the management of intrahepatic cholangiocarcinoma with FGFR2 gene alterations.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

In some aspects, the techniques described herein relate to a method of treating cholangiocarcinoma in a subject with cirrhosis and total bilirubin >1.5 times the upper limit of normal up to 3 times the upper limit of normal, comprising orally administering to the subject 20 mg of futibatinib once a day.

In some aspects, the techniques described herein relate to a method of treating cholangiocarcinoma in a subject with cirrhosis and total bilirubin >1.0 to 1.5 times the upper limit of normal, comprising orally administering to the subject, futibatinib at a dose of 20 mg daily.

In some aspects, the techniques described herein relate to a method of treating previously treated, unresectable, locally advanced or metastatic cholangiocarcinoma harboring fibroblast growth factor receptor 2 gene fusions or other rearrangement in a subject with cirrhosis and total bilirubin >1.5 times the upper limit of normal to 3 times the upper limit of normal, comprising orally administering to the subject 20 mg of futibatinib once a day, wherein the dose is not reduced compared with a standard dose for a subject with normal hepatic function and wherein a mean Cₘₐₓ of unbound futibatinib and mean AUC of unbound futibatinib in the subject after administration of the futibatinib is increased compared with a mean Cₘₐₓ of unbound futibatinib and a mean AUC of unbound futibatinib of a subject with normal hepatic function after administration of 20 mg of futibatinib once daily.

In some aspects, the techniques described herein relate to a method of treating cholangiocarcinoma in a subject with moderate to severe hepatic impairment as defined by Child-Pugh B to C and with total bilirubin <1.5 times the upper limit of normal comprising orally administering to the subject 20 mg of futibatinib once a day.

The foregoing general description of the illustrative embodiments and the following detailed description thereof are merely exemplary aspects of the teachings of this disclosure and are not restrictive.

### DETAILED DESCRIPTION

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof Δny listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Where a range of values is provided, it is intended that each intervening value between the upper and lower limit of that range and any other stated or intervening value in that stated range is encompassed within the disclosure. For example, if a range of 1 mm to 8 mm is stated, it is intended that 2 mm, 3 mm, 4 mm, 5 mm, 6 mm, and 7 mm are also explicitly disclosed.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

As used herein, the term “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation.

The terms “administer,” “administering” or “administration” as used herein refer to directly administering a compound or a composition to a subject.

As used herein, the term “composition” refers to a mixture or combination of two or more components, substances, or ingredients that together form a product with specific properties or characteristics. In some embodiments, a composition may comprise futibatinib as an active pharmaceutical ingredient (API) combined with one or more pharmaceutically acceptable excipients, carriers, diluents, stabilizers, preservatives, solubilizers, surfactants, disintegrants, binders, lubricants, or other additives that facilitate the manufacture, stability, bioavailability, or administration of the formulation. The pharmaceutical composition containing futibatinib may be formulated in various dosage forms including but not limited to tablets, capsules, powders, solutions, suspensions, emulsions, or other suitable forms for oral administration. The specific components and their relative proportions in the composition are selected to optimize the delivery, absorption, and therapeutic efficacy of futibatinib while maintaining acceptable stability and safety profiles.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention.

As used herein, the term “Cholangiocarcinoma” refers to a type of cancer that forms in the bile ducts. In some cases, cholangiocarcinoma may be classified as intrahepatic, perihilar, or distal based on the location of origin within the biliary system.

As used herein, the term “intrahepatic cholangiocarcinoma” refers to cholangiocarcinoma that originates in the bile ducts inside the liver.

As used herein, the term “unresectable” refers to a tumor or cancer that cannot be removed completely through surgery.

As used herein, the term “locally advanced” refers to cancer that has spread from where it started to nearby tissue or lymph nodes.

As used herein, the term “metastatic” refers to cancer that has spread from its original site to other parts of the body.

As used herein, the term “fibroblast growth factor receptor 2 (FGFR2)” refers to a protein that is a member of the fibroblast growth factor receptor family. In some cases, FGFR2 may be involved in cell division, growth, and differentiation.

As used herein, the term “FGFR2 gene fusion” refers to a genetic alteration in which part of the FGFR2 gene is abnormally connected to another gene, potentially leading to overactive FGFR2 signaling.

As used herein, the term “FGFR2 gene rearrangement” refers to a structural change in the FGFR2 gene that may alter its function or expression.

As used herein, the term “futibatinib” refers to a small molecule inhibitor of FGFR. Futibatinib is also known by the brand name LYTGOBI™. Chemically, futibatinib is 2-(4-{4-[(3,3-dimethylbutyl)amino]-7-methoxy-1,6-naphthyridin-5-yl}phenyl)-1,3,4-oxadiazole. It has a molecular formula of C25H27N5O2 and a molecular weight of 429.52 g/mol. The chemical structure consists of a 1,6-naphthyridine core with a methoxy group at position 7, an amino group linked to a 3,3-dimethylbutyl chain at position 4, a phenyl group at position 5, and a 1,3,4-oxadiazole group attached to the phenyl ring. In some cases, futibatinib may be used to treat certain types of cancer. Futibatinib has the following structure:

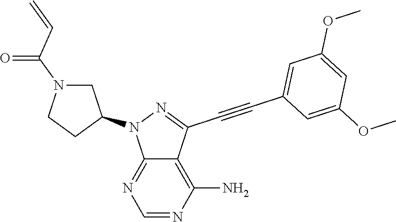 [SMILES: C=CC(=O)N1CC[C@H](n2nc(C#Cc3cc(OC)cc(OC)c3)c3c(N)ncnc32)C1]

As used herein, the term “cirrhosis” refers to a late stage of scarring (fibrosis) of the liver caused by many forms of liver diseases and conditions.

As used herein, the term “total bilirubin” refers to a measure of the total amount of bilirubin in the blood, including both conjugated and unconjugated forms.

As used herein, the term “upper limit of normal (ULN)” refers to the highest value of a laboratory test result that is considered within the normal range for a healthy individual. The normal range for AST is 13-39 IU/L, therefore the ULN of AST is 39 IU/L. The total bilirubin normal range is 0.3-1 mg/dL, therefore the ULN of total bilirubin is 1 mg/dL.

As used herein, the term “Child-Pugh score” refers to a scoring system used to assess the prognosis of chronic liver disease, mainly cirrhosis. Child-Pugh classification widely used and accepted by regulatory agencies (including the US FDA [FDA 2003] and the European Medicines Agency [EMA; EMA 2005]). The Child-Pugh score may be used to help assess liver function, predict prognosis, and guide treatment decisions in patients with chronic liver disease. The scale employs 5 clinical measures of liver disease listed in Table 1. Each measure is scored 1 to 3, with 3 indicating most severe derangement. The bilirubin score in the table is dependent upon the type of cirrhosis (primary biliary cirrhosis versus all other causes). Subjects' scores of 5 to 6, 7 to 9, and 10 to 15 on this scale are classified as having mild (Child Pugh-A), moderate (Child Pugh-B), and severe (Child Pugh-C) hepatic failure, respectively.

TABLE 1
Derivation of Child-Pugh Classification Score. Adapted from
FDA Guidance for Industry. Pharmacokinetics in Patients
with Impaired Hepatic Function: Study Design, Data Analysis,
and Impact on Dosing and Labeling (FDA 2003).

| Parameter | 1 point | 2 Points | 3 Points |
|---|---|---|---|
| Serum albumin (g/dL) | >3.5 | 2.8 to 3.5 | <2.8 |
| Total serum bilirubin | <2.0 | 2.0 to 3.0 | >3.0 |
| (mg/dL) | | | |
| Prothrombin time (PT) | <4 | 4 to 6 | >6 |
| (seconds prolonged) or | <1.70 | 1.70 to 2.30 | >2.30 |
| international normalized | | | |
| ratio (INR) | | | |
| Ascites | Absent | Slight | Moderate or subject |
| | | | on medication(s) to |
| | | | control ascites |
| Hepatic encephalopathy | None | Grade 1 or 2 | Grade 3 or 4 or |
| | | | subject receiving |
| | | | medication(s) to |
| | | | prevent |
| | | | encephalopathy |

As used herein, the term “Child-Pugh A” may refer to a total score of 5-6 points, indicating relatively preserved liver function or potentially mild hepatic impairment.

As used herein, the term “Child-Pugh B” may refer to a total score of 7-9 points, suggesting moderate hepatic impairment.

As used herein, the term “Child-Pugh C” may refer to a total score of 10-15 points, indicating severe hepatic impairment.

As used herein, the term “hepatic encephalopathy” refers to is a neurological complication of liver disease where the brain's function is impaired due to the buildup of toxins in the blood that the liver can no longer filter out. This condition can lead to a range of symptoms, from mild confusion and memory problems to severe disorientation, confusion, and even coma. Hepatic encephalopathy scores used to determine Child-Pugh scores are outlined in Table 2.

TABLE 2
Determination of Encephalopathy Grade. Adapted from FDA Guidance for Industry.
Pharmacokinetics in Patients with Impaired Hepatic Function: Study Design,
Data Analysis, and Impact on Dosing and Labeling (FDA 2003).

| Encephalopathy | Definition |
|---|---|
| 0 | Normal consciousness, personality, neurological exam |
| 1 | Restless, sleep disturbed, irritable/agitated, tremor, impaired |
| | handwriting |
| 2 | Lethargic, time-disoriented, inappropriate, asterixis, ataxia |
| 3^{a} | Somnolent, stuporous, place-disoriented, hyperactive reflexes, |
| | rigidity |
| 4^{a} | Unarousable coma, no personality/behavior, decerebrate |

^{a}Subjects with clinically active Grade 3 or 4 encephalopathy were excluded from these studies.

As used herein, the term “NCI-ODWG criteria” or “NCI Organ Dysfunction Working Group criteria” refers to a classification system developed by the National Cancer Institute Organ Dysfunction Working Group for categorizing hepatic impairment in cancer patients. This system uses serum total bilirubin and aspartate aminotransferase (AST) levels to classify hepatic function into four categories: normal, mild dysfunction, moderate dysfunction, and severe dysfunction. In some cases, the NCI-ODWG criteria is used to guide dose adjustments for cancer therapies in patients with varying degrees of liver impairment. The specific cutoff values for each category as used herein is defined as follows:

Normal hepatic function: total bilirubin and AST≤upper limit of normal (ULN)

Mild hepatic dysfunction: total bilirubin >1.0 to 1.5×ULN (any AST) or AST>ULN and total bilirubin ≤ULN.

Moderate hepatic dysfunction: total bilirubin >1.5 to 3×ULN (any AST)

Severe hepatic dysfunction: total bilirubin >3×ULN (any AST)

As used herein, the term “moderate hepatic impairment” refers to a level of liver dysfunction that is significant but not severe. In some cases, this may be defined by specific clinical parameters such as Child-Pugh B classification.

As used herein, the term “severe hepatic impairment” may refer to a high level of liver dysfunction. In some cases, this may be defined by specific clinical parameters such as Child-Pugh C classification.

As used herein, the term “previously treated” refers to a subject who has received prior therapy for their condition. In some cases, this may include but is not limited to surgery, radiation therapy, chemotherapy, or targeted therapy.

As used herein, the term “subject” refers to any living organism that may receive medical treatment, participate in a clinical study, or be the focus of scientific research. In some cases, a subject may be a human patient, a healthy volunteer, or an animal model used in preclinical studies. The term “subject” may encompass individuals of any age, gender, health status, or genetic background who are candidates for diagnostic evaluation, therapeutic intervention, or experimental investigation.

As used herein, the term “healthy subject” refers to an individual who does not have any known significant medical conditions, diseases, or disorders that may affect the results of a study or treatment. In some cases, a healthy subject may be free from clinically relevant abnormalities in medical history, physical examination, vital signs, laboratory tests, and other assessments as determined by the investigator. The term “healthy subject” may be used in clinical trials or research studies as a control group or to establish baseline parameters for comparison with subjects who have specific medical conditions.

In some embodiments the term “mean” can refer to mean, geometric mean, or geometric least square mean. In certain embodiments the term “mean Cₘₐₓ” can refer to mean Cₘₐₓ, geometric mean Cₘₐₓ, or geometric least square mean Cₘₐₓ. In certain embodiments the term “mean unbound futibatinib Cₘₐₓ” can refer to mean unbound futibatinib Cₘₐₓ, geometric mean unbound futibatinib Cₘₐₓ, or geometric least square mean unbound futibatinib Cₘₐₓ. In certain embodiments the term “mean total futibatinib Cₘₐₓ” can refer to mean total futibatinib Cₘₐₓ, geometric mean total futibatinib Cₘₐₓ, or geometric least square mean total futibatinib Cₘₐₓ. In certain embodiments the term “mean AUC” can refer to mean AUC, geometric mean AUC, or geometric least square mean AUC. In certain embodiments the term “mean unbound futibatinib AUC” can refer to mean unbound futibatinib AUC, geometric mean unbound futibatinib AUC, or geometric least square mean unbound futibatinib AUC. In certain embodiments the term “mean total futibatinib AUC” can refer to mean total futibatinib AUC, geometric mean total futibatinib AUC, or geometric least square mean total futibatinib AUC.

### INTRODUCTION

Embodiments of the present disclosure are directed to methods of treating cholangiocarcinoma using futibatinib in subjects with various degrees of hepatic impairment. In some embodiments, the methods involve orally administering futibatinib to subjects with cirrhosis and elevated total bilirubin levels. The subjects may have previously treated, unresectable, locally advanced or metastatic intrahepatic cholangiocarcinoma harboring fibroblast growth factor receptor 2 (FGFR2) gene fusions or other rearrangements.

In some embodiments, the methods are directed to treating cholangiocarcinoma in subjects with moderate to severe hepatic impairment, as defined by Child-Pugh B to C classification, and with total bilirubin below certain thresholds. Other embodiments are directed to treating cholangiocarcinoma in subjects with cirrhosis and total bilirubin levels exceeding specific multiples of the upper limit of normal.

The methods described herein may involve administering futibatinib at a dose of about 20 mg once daily. In certain embodiments, this dose is not reduced compared to the standard dose for subjects with normal hepatic function, despite the presence of hepatic impairment in the treated subjects.

Some embodiments of the methods include monitoring subjects for increased incidence or severity of adverse reactions compared to subjects with normal hepatic function. The methods may also involve monitoring for specific conditions such as hyperphosphatemia and initiating appropriate therapies if certain thresholds are exceeded.

The embodiments described herein provide approaches for treating cholangiocarcinoma with futibatinib in subjects with varying degrees of hepatic impairment, while considering potential changes in drug exposure and the need for monitoring specific parameters.

### Methods Of Treatment

Embodiments of the present disclosure are directed to methods of treating cholangiocarcinoma in subjects with various degrees of hepatic impairment.

Subject with Cirrhosis and Total Bilirubin >1.5 Times the Upper Limit of Normal Up to 3 Times the Upper Limit of Normal

Some embodiments describe a method of treating cholangiocarcinoma in a subject with cirrhosis and total bilirubin >1.5 times the upper limit of normal up to 3 times the upper limit of normal, comprising orally administering to the subject 20 mg of futibatinib once a day.

In some embodiments, the subject has previously treated, unresectable, locally advanced or metastatic intrahepatic cholangiocarcinoma harboring fibroblast growth factor receptor 2 (FGFR2) gene fusions or other rearrangements.

In some embodiments, a mean Cₘₐₓ of unbound futibatinib in the subject after administration of the futibatinib is increased compared to a mean Cₘₐₓ of unbound futibatinib of a subject with normal hepatic function after administration of about 20 mg of futibatinib once daily. The increase in geometric least square mean (geometric LSM) Cₘₐₓ of unbound futibatinib in some embodiments is about 3.2-fold compared to the geometric LSM Cₘₐₓ of unbound futibatinib in a subject with normal hepatic function after administration of 20 mg of futibatinib once daily. In some embodiments, the geometric LSM Cₘₐₓ of unbound futibatinib in a subject with normal hepatic function after administration of about 20 mg of futibatinib once daily is about 2.06 ng/mL. In some embodiments the subject with normal hepatic function is a subject that does not have cholangiocarcinoma. In some embodiments the subject with normal hepatic function is a healthy subject.

In some embodiments, a mean Cₘₐₓ of unbound futibatinib in the subject after administration of the futibatinib is increased compared to a mean Cₘₐₓ of unbound futibatinib of a subject with normal hepatic function after administration of about 20 mg of futibatinib once daily. The increase in the mean Cₘₐₓ of unbound futibatinib in some embodiments is about 3.2-fold compared to the mean Cₘₐₓ of unbound futibatinib in a subject with normal hepatic function after administration of 20 mg of futibatinib once daily. In some embodiments, the mean Cₘₐₓ of unbound futibatinib in a subject with normal hepatic function after administration of about 20 mg of futibatinib once daily is about 2.06 ng/mL. In some embodiments the subject with normal hepatic function is a subject that does not have cholangiocarcinoma. In some embodiments the subject with normal hepatic function is a healthy subject.

In some embodiments, a mean AUC of unbound futibatinib in the subject after administration of the futibatinib is increased compared to a mean AUC of unbound futibatinib of a subject with normal hepatic function after administration of about 20 mg of futibatinib once daily. The increase in geometric LSM AUC of unbound futibatinib in some embodiments is about 2.4-fold compared to the geometric LSM AUC of unbound futibatinib in a subject with normal hepatic function after administration of 20 mg of futibatinib once daily. In some embodiments, the geometric LSM AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

In some embodiments, a mean AUC of unbound futibatinib in the subject after administration of the futibatinib is increased compared to a mean AUC of unbound futibatinib of a subject with normal hepatic function after administration of about 20 mg of futibatinib once daily. The increase in the mean AUC of unbound futibatinib in some embodiments is about 2.4-fold compared to the mean AUC of unbound futibatinib in a subject with normal hepatic function after administration of 20 mg of futibatinib once daily. In some embodiments, the mean AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

In some embodiments, both the mean Cₘₐₓ of unbound futibatinib and the mean AUC of unbound futibatinib in the subject after administration of the futibatinib is increased compared to the mean Cₘₐₓ of unbound futibatinib and the mean AUC of unbound futibatinib of a subject with normal hepatic function after administration of about 20 mg of futibatinib once daily. The increase in geometric LSM Cₘₐₓ of unbound futibatinib in some embodiments is about 3.2-fold compared to the geometric LSM Cₘₐₓ of unbound futibatinib in a subject with normal hepatic function after administration of 20 mg of futibatinib once daily. The increase in mean AUC of unbound futibatinib in some embodiments is about 2.4-fold compared to the geometric LSM AUC of unbound futibatinib in a subject with normal hepatic function after administration of 20 mg of futibatinib once daily. In some embodiments, the geometric LSM Cₘₐₓ of unbound futibatinib in a subject with normal hepatic function after administration of about 20 mg of futibatinib once daily is about 2.06 ng/mL. In some embodiments, the geometric LSM AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

In some embodiments, both the mean Cₘₐₓ of unbound futibatinib and the mean AUC of unbound futibatinib in the subject after administration of the futibatinib is increased compared to the mean Cₘₐₓ of unbound futibatinib and the mean AUC of unbound futibatinib of a subject with normal hepatic function after administration of about 20 mg of futibatinib once daily. The increase in the mean Cₘₐₓ of unbound futibatinib in some embodiments is about 3.2-fold compared to the mean Cₘₐₓ of unbound futibatinib in a subject with normal hepatic function after administration of 20 mg of futibatinib once daily. The increase in mean AUC of unbound futibatinib in some embodiments is about 2.4-fold compared to the mean AUC of unbound futibatinib in a subject with normal hepatic function after administration of 20 mg of futibatinib once daily. In some embodiments, the mean Cₘₐₓ of unbound futibatinib in a subject with normal hepatic function after administration of about 20 mg of futibatinib once daily is about 2.06 ng/mL. In some embodiments, the mean AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

In some embodiments, the subject is monitored for an increased risk of one or more adverse reactions compared to risk of one or more adverse reactions in a subject with normal hepatic function, wherein the increased risk of one or more adverse reactions is attributable to higher unbound futibatinib in the plasma of the subject. In some embodiments, the subject is monitored for an increased risk of one or more adverse reactions compared to the risk of one or more adverse reactions in a healthy subject with normal hepatic function, wherein the increased risk of one or more adverse reactions is attributable to higher unbound futibatinib in the plasma of the subject.

In some embodiments, the adverse reaction is selected from the group consisting of skin and subcutaneous tissue disorders, gastrointestinal disorders, fatigue, metabolism and nutrition disorders, musculoskeletal and connective tissue disorders, eye disorders, nervous system disorders, infections, weight decrease, and combinations thereof.

In some embodiments, the adverse reaction is selected from the group consisting of nail toxicity, nail disorder, nail discoloration, nail dystrophy, nail hypertrophy, nail infection, nail pigmentation, onychalgia, onychoclasis, onycholysis, onychomadesis, onychomycosis, paronychia, alopecia, dry skin, palmar-plantar erythrodysesthesia syndrome, constipation, diarrhea, colitis, gastroenteritis, dry mouth, stomatitis, glossitis, mouth ulceration, mucosal inflammation, pharyngeal inflammation, tongue ulceration, abdominal pain, abdominal discomfort, abdominal pain upper, gastrointestinal pain, hepatic pain, nausea, vomiting, hematemesis, fatigue, asthenia, decreased appetite, musculoskeletal pain, back pain, bone pain, musculoskeletal chest pain, musculoskeletal discomfort, musculoskeletal stiffness, myalgia, neck pain, non-cardiac chest pain, pain in extremity, spinal pain, arthralgia, arthritis, dry eye, keratitis, lacrimation increased, photokeratitis, punctate keratitis, ulcerative keratitis, dysgeusia, ageusia, taste disorder, urinary tract infection, cystitis, dysuria, weight decrease and any combination thereof.

In some embodiments, the adverse reaction is selected from the group consisting of pyrexia, gastrointestinal hemorrhage, ascites, musculoskeletal pain, bile duct obstruction and combinations thereof.

The occurrence of these adverse reactions may be monitored in subjects receiving futibatinib treatment, particularly in those with hepatic impairment. In certain cases, the incidence or severity of these adverse reactions may be compared to that observed in subjects with normal hepatic function to assess any potential increased risk associated with hepatic dysfunction. The monitoring of these specific adverse reactions may help guide treatment decisions and patient management strategies in subjects with cholangiocarcinoma and varying degrees of hepatic impairment.

In some embodiments, the risk of one or more adverse reactions in a subject with normal hepatic function is defined in Table A:

TABLE A

| | All Grades | Grade 3 |
|---|---|---|
| Adverse Reaction | (%)* | (%)* |
| One or more of nail toxicity, nail disorder, nail discoloration, | 47 | 1.9 |
| nail dystrophy, nail hypertrophy, nail infection, nail | | |
| pigmentation, onychalgia, onychoclasis, onychomadesis, | | |
| onychomycosis, or paronychia | | |
| Alopecia | 34 | 0 |
| Dry skin | 29 | 0 |
| Palmar-plantar erythrodysesthesia syndrome | 21 | 4.9 |
| Constipation | 39 | 0 |
| One or more of diarrhea, colitis, or gastroenteritis | 39 | 1 |
| Dry mouth | 35 | 0 |
| One or more of stomatitis, glossitis, mouth ulceration, mucosal | 30 | 6 |
| inflammation, pharyngeal inflammation, or tongue ulceration | | |
| One or more of abdominal pain, abdominal discomfort, | 30 | 2.9 |
| abdominal pain upper, gastrointestinal pain, or hepatic pain | | |
| Nausea | 24 | 1.9 |
| One or more of vomiting or hematemesis | 20 | 1 |
| One or more of fatigue or asthenia | 37 | 8 |
| Decreased appetite | 23 | 2.9 |
| One or more of musculoskeletal pain back pain, bone pain, | 43 | 3.9 |
| musculoskeletal chest pain, musculoskeletal discomfort, | | |
| musculoskeletal pain, musculoskeletal stiffness, myalgia, neck | | |
| pain, non-cardiac chest pain, pain in extremity, or spinal pain | | |
| One or more of arthralgia or arthritis i | 25 | 0 |
| One or more of dry eye, keratitis, lacrimation increased, | 25 | 1 |
| photokeratitis, punctate keratitis, or ulcerative keratitis | | |
| One or more of dysgeusia, ageusia, and taste disorder | 25 | 0 |
| One or more of urinary tract infection, cystitis, or dysuria 1 | 23 | 2.9 |
| Weight decreased | 18 | 3.9 |

*per NCI CTCAE 4.03

In some embodiments, the risk of one or more adverse reactions in a subject with normal hepatic function is defined in Table B:

TABLE B

| Adverse Reaction | Grade 3 (%)* |
|---|---|
| pyrexia | 3.9 |
| gastrointestinal hemorrhage | 3.9 |
| Ascites | 2.9 |
| musculoskeletal pain | 2.9 |
| bile duct obstruction | 2.9 |

*per NCI CTCAE 4.03

In some embodiments, the increased risk of one or more adverse reactions may be attributed to an increased mean Cₘₐₓ of unbound futibatinib in the subject after administration of the futibatinib compared with a mean Cₘₐₓ of unbound futibatinib in a subject with normal hepatic function. The higher Cₘₐₓ can result in greater peak exposure to the drug, which may lead to an increased likelihood of concentration-dependent adverse effects. In certain cases, this increased Cₘₐₓ may be due to altered drug distribution or reduced hepatic clearance in subjects with impaired liver function.

In some embodiments, the increased risk of adverse reactions can be related to an elevated mean AUC of unbound futibatinib in the subject compared to a subject with normal hepatic function. The higher AUC may indicate prolonged exposure to the drug, potentially leading to cumulative effects and an increased risk of time-dependent toxicities. This increased AUC can, in some instances, be attributed to reduced drug metabolism or elimination in subjects with hepatic impairment.

The method may, in some embodiments, involve monitoring for adverse reactions based on both increased mean Cₘₐₓ and mean AUC of unbound futibatinib in the subject. The combination of higher peak concentrations and prolonged exposure can potentially lead to a synergistic increase in the risk of adverse events. This may be particularly relevant for adverse reactions that are sensitive to both the intensity and duration of drug exposure.

In some embodiments, the geometric LSM Cₘₐₓ of unbound futibatinib in the subject can be increased by approximately 3.2-fold compared to a subject with normal hepatic function. In some embodiments, the mean Cₘₐₓ of unbound futibatinib in the subject can be increased by approximately 3.2-fold compared to a subject with normal hepatic function. This substantial increase in peak concentration may result in a proportional increase in the risk of concentration-dependent adverse reactions. The magnitude of this increase can vary among individuals and may depend on factors such as the severity of hepatic impairment or concomitant medications.

Some embodiments may involve situations where the geometric LSM AUC of unbound futibatinib in the subject is increased by about 2.4-fold compared to a subject with normal hepatic function. Some embodiments may involve situations where the mean AUC of unbound futibatinib in the subject is increased by about 2.4-fold compared to a subject with normal hepatic function. This increase in total drug exposure over time can potentially lead to a higher risk of cumulative toxicities or adverse reactions that develop with prolonged drug exposure. The extent of this increase may vary based on individual patient factors and the specific nature of their hepatic impairment.

In some cases, the method can involve comparing the observed Cₘₐₓ and AUC values to reference values in subjects with normal hepatic function. For instance, a geometric LSM Cₘₐₓ of unbound futibatinib in a subject with normal hepatic function may be about 2.06 ng/mL, while a geometric LSM AUC may be about 7.53 ng*hr/mL. Deviations from these reference values can help quantify the degree of altered pharmacokinetics in subjects with hepatic impairment and may guide monitoring strategies for adverse reactions.

The method may, in some embodiments, include assessing the relationship between the magnitude of increase in Cₘₐₓ or AUC and the severity or frequency of adverse reactions. This can help establish exposure-response relationships and potentially identify threshold values above which the risk of specific adverse events significantly increases.

In certain embodiments, the method can involve monitoring for specific adverse reactions known to be associated with higher drug exposure, such as hyperphosphatemia or eye disorders. The frequency and intensity of monitoring may be adjusted based on the degree of increase in Cₘₐₓ and AUC compared to subjects with normal hepatic function.

Some embodiments may include strategies to mitigate the increased risk of adverse reactions associated with higher Cₘₐₓ and AUC. These strategies can comprise dose adjustments, altered dosing schedules, or implementation of more frequent safety assessments in subjects with significantly elevated exposure to unbound futibatinib.

The method may, in some cases, involve pharmacokinetic modeling to predict individual patient exposure based on their hepatic function status and other relevant factors. This approach can help personalize monitoring strategies and identify subjects who may be at particularly high risk of adverse reactions due to increased Cₘₐₓ and AUC of unbound futibatinib.

Some embodiments describe a method of treating previously treated, unresectable, locally advanced or metastatic cholangiocarcinoma harboring fibroblast growth factor receptor 2 gene fusions or other rearrangement in a subject with cirrhosis and total bilirubin >1.5 times the upper limit of normal to 3 times the upper limit of normal, comprising orally administering to the subject 20 mg of futibatinib once a day, wherein the dose is not reduced compared with a standard dose for a subject with normal hepatic function and wherein a mean Cₘₐₓ of unbound futibatinib and mean AUC of unbound futibatinib in the subject after administration of the futibatinib is increased compared with a mean Cₘₐₓ of unbound futibatinib and a mean AUC of unbound futibatinib of a subject with normal hepatic function after administration of 20 mg of futibatinib once daily.

The increase in geometric LSM Cₘₐₓ of unbound futibatinib in some embodiments is about 3.2-fold compared to the geometric LSM Cₘₐₓ of unbound futibatinib in a subject with normal hepatic function after administration of 20 mg of futibatinib once daily. The increase in geometric LSM AUC of unbound futibatinib in some embodiments is about 2.4-fold compared to the geometric LSM AUC of unbound futibatinib in a subject with normal hepatic function after administration of 20 mg of futibatinib once daily. In some embodiments, the geometric LSM Cₘₐₓ of unbound futibatinib in a subject with normal hepatic function after administration of about 20 mg of futibatinib once daily is about 2.06 ng/mL. In some embodiments, the geometric LSM AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

The increase in mean Cₘₐₓ of unbound futibatinib in some embodiments is about 3.2-fold compared to the mean Cₘₐₓ of unbound futibatinib in a subject with normal hepatic function after administration of 20 mg of futibatinib once daily. The increase in mean AUC of unbound futibatinib in some embodiments is about 2.4-fold compared to the mean AUC of unbound futibatinib in a subject with normal hepatic function after administration of 20 mg of futibatinib once daily. In some embodiments, the mean Cₘₐₓ of unbound futibatinib in a subject with normal hepatic function after administration of about 20 mg of futibatinib once daily is about 2.06 ng/mL. In some embodiments, the mean AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

In some embodiments, the subject is monitored for an increased risk of one or more adverse reactions compared to risk of one or more adverse reactions in a subject with normal hepatic function, wherein the increased risk of one or more adverse reactions is attributable to higher unbound futibatinib in the plasma of the subject. In some embodiments, the subject is monitored for an increased risk of one or more adverse reactions compared to the risk of one or more adverse reactions in a healthy subject with normal hepatic function, wherein the increased risk of one or more adverse reactions is attributable to higher unbound futibatinib in the plasma of the subject.

In some embodiments, the subject does not have a Child-Pugh score designation of A.

Subject with Cirrhosis and Total Bilirubin >1.0 to 1.5 Times the Upper Limit of Normal

Some embodiments describe a method of treating cholangiocarcinoma in a subject with cirrhosis and total bilirubin >1.0 to 1.5 times the upper limit of normal, comprising orally administering to the subject, futibatinib at a dose of 20 mg daily.

In some embodiments, the subject has previously treated, unresectable, locally advanced or metastatic cholangiocarcinoma harboring fibroblast growth factor receptor 2 gene fusions or other rearrangement. In some embodiments, the dose is not reduced compared with a standard dose for a subject with normal hepatic function.

Some embodiments, describe a method of treating previously treated, unresectable, locally advanced or metastatic cholangiocarcinoma harboring fibroblast growth factor receptor 2 gene fusions or other rearrangement in a subject with cirrhosis and total bilirubin >1.0 to 1.5 times the upper limit of normal, comprising orally administering to the subject, futibatinib at a dose of about 20 mg daily, wherein the dose is not reduced compared to a standard dose for a subject with normal hepatic function.

In some embodiments, a mean Cₘₐₓ of unbound futibatinib in the subject after administration of the futibatinib is increased compared to a mean Cₘₐₓ of unbound futibatinib of a subject with normal hepatic function after administration of about 20 mg of futibatinib once daily. The increase in geometric LSM Cₘₐₓ of unbound futibatinib in some embodiments is about 1.7-fold compared to the geometric LSM Cₘₐₓ of unbound futibatinib in a subject with normal hepatic function after administration of 20 mg of futibatinib once daily. In some embodiments, the geometric LSM Cₘₐₓ of unbound futibatinib in a subject with normal hepatic function after administration of about 20 mg of futibatinib once daily is about 2.06 ng/mL. In some embodiments the subject with normal hepatic function is a subject that does not have cholangiocarcinoma. In some embodiments the subject with normal hepatic function is a healthy subject.

In some embodiments, a mean Cₘₐₓ of unbound futibatinib in the subject after administration of the futibatinib is increased compared to a mean Cₘₐₓ of unbound futibatinib of a subject with normal hepatic function after administration of about 20 mg of futibatinib once daily. The increase in the mean Cₘₐₓ of unbound futibatinib in some embodiments is about 1.7-fold compared to the mean Cₘₐₓ of unbound futibatinib in a subject with normal hepatic function after administration of 20 mg of futibatinib once daily. In some embodiments, the mean Cₘₐₓ of unbound futibatinib in a subject with normal hepatic function after administration of about 20 mg of futibatinib once daily is about 2.06 ng/mL. In some embodiments the subject with normal hepatic function is a subject that does not have cholangiocarcinoma. In some embodiments the subject with normal hepatic function is a healthy subject.

In some embodiments, a mean AUC of unbound futibatinib in the subject after administration of the futibatinib is increased compared to a mean AUC of unbound futibatinib of a subject with normal hepatic function after administration of about 20 mg of futibatinib once daily. The increase in geometric LSM AUC of unbound futibatinib in some embodiments is about 1.7-fold compared to the geometric LSM AUC of unbound futibatinib in a subject with normal hepatic function after administration of 20 mg of futibatinib once daily. In some embodiments, the geometric LSM AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

In some embodiments, a mean AUC of unbound futibatinib in the subject after administration of the futibatinib is increased compared to a mean AUC of unbound futibatinib of a subject with normal hepatic function after administration of about 20 mg of futibatinib once daily. The increase in the mean AUC of unbound futibatinib in some embodiments is about 1.7-fold compared to the mean AUC of unbound futibatinib in a subject with normal hepatic function after administration of 20 mg of futibatinib once daily. In some embodiments, the mean AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

In some embodiments, both the mean Cₘₐₓ of unbound futibatinib and the mean AUC of unbound futibatinib in the subject after administration of the futibatinib is increased compared to the mean Cₘₐₓ of unbound futibatinib and the mean AUC of unbound futibatinib of a subject with normal hepatic function after administration of about 20 mg of futibatinib once daily. The increase in geometric LSM Cₘₐₓ of unbound futibatinib in some embodiments is about 1.7-fold compared to the geometric LSM Cₘₐₓ of unbound futibatinib in a subject with normal hepatic function after administration of 20 mg of futibatinib once daily. The increase in geometric LSM AUC of unbound futibatinib in some embodiments is about 1.7-fold compared to the geometric LSM AUC of unbound futibatinib in a subject with normal hepatic function after administration of 20 mg of futibatinib once daily. In some embodiments, the geometric LSM Cₘₐₓ of unbound futibatinib in a subject with normal hepatic function after administration of about 20 mg of futibatinib once daily is about 2.06 ng/mL. In some embodiments, the geometric LSM AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

In some embodiments, both the mean Cₘₐₓ of unbound futibatinib and the mean AUC of unbound futibatinib in the subject after administration of the futibatinib is increased compared to the mean Cₘₐₓ of unbound futibatinib and the mean AUC of unbound futibatinib of a subject with normal hepatic function after administration of about 20 mg of futibatinib once daily. The increase in the mean Cₘₐₓ of unbound futibatinib in some embodiments is about 1.7-fold compared to the mean Cₘₐₓ of unbound futibatinib in a subject with normal hepatic function after administration of 20 mg of futibatinib once daily. The increase in the mean AUC of unbound futibatinib in some embodiments is about 1.7-fold compared to the mean AUC of unbound futibatinib in a subject with normal hepatic function after administration of 20 mg of futibatinib once daily. In some embodiments, the mean Cₘₐₓ of unbound futibatinib in a subject with normal hepatic function after administration of about 20 mg of futibatinib once daily is about 2.06 ng/mL. In some embodiments, the mean AUC of unbound futibatinib in a subject with normal hepatic function after administration of about 20 mg of futibatinib once daily is about 7.53 ng*hr/mL. In some embodiments the subject with normal hepatic function is a subject that does not have cholangiocarcinoma. In some embodiments the subject with normal hepatic function is a healthy subject.

In some embodiments, the subject does not have a Child-Pugh score designation of A.

Subjects with Moderate to Severe Hepatic Impairment as Defined by Child-Pugh B to C and with Total Bilirubin <1.5 Times the Upper Limit of Normal

In some embodiments, the method includes treating cholangiocarcinoma in a subject with moderate to severe hepatic impairment as defined by Child-Pugh B to C and with total bilirubin <1.5 times the upper limit of normal comprising orally administering to the subject about 20 mg of futibatinib once a day. In some embodiments, the method includes treating cholangiocarcinoma in a subject with moderate to severe hepatic impairment as defined by Child-Pugh B to C and with total bilirubin <1.5 times the upper limit of normal to about 3 times the upper limit of normal comprising orally administering to the subject about 20 mg of futibatinib once per day.

In some embodiments, the subject is monitored for hyperphosphatemia and appropriate phosphate lowering therapy is initiated if serum phosphate is ≥5.5 mg/dL. Initiate a low phosphate diet and phosphate lowering therapy when serum phosphate level is ≥5.5 mg/dL. For serum phosphate levels >7 mg/dL, initiate or intensify phosphate lowering therapy and dose reduce, withhold, or permanently discontinue administration of futibatinib based on duration and severity of hyperphosphatemia

In some embodiments, the subject is monitored by comprehensive ophthalmological examination including optical coherence tomography (OCT) prior to initiation of therapy, every 2 months for the first 6 months, and every 3 months thereafter.

In some embodiments, the subject does not have a Child-Pugh score designation of A.

In any embodiment described herein, the administration of futibatinib is continued until disease progression or unacceptable toxicity occurs. The duration of treatment is monitored and adjusted based on individual patient response and tolerability.

In any embodiment described herein, dose modifications may be implemented for retinal pigment epithelial detachment (RPED), the dose is modified based on the severity and persistence of the condition. In some cases, futibatinib is withheld for Grade 2 RPED lasting more than 14 days or for any Grade 3 RPED. Treatment is resumed at a reduced dose when RPED improves to Grade 0 or 1.

In any embodiment described herein, dose modifications may be implemented for hyperphosphatemia, dose modifications are based on serum phosphate levels. In some embodiments, a low phosphate diet and phosphate lowering therapy is initiated when serum phosphate level is ≥5.5 mg/dL. For serum phosphate levels >7 mg/dL, initiate or intensify phosphate lowering therapy and dose reduce, withhold, or permanently discontinue administration of futibatinib based on duration and severity of hyperphosphatemia.

In some embodiments, concomitant use of dual P-glycoprotein (P-gp) and strong cytochrome P450 3A (CYP3A) inhibitors or inducers with futibatinib is avoided. This precaution helps prevent potential drug interactions that could affect the pharmacokinetics of futibatinib.

In some embodiments, more frequent monitoring for adverse reactions is implemented when futibatinib is administered concomitantly with drugs that are sensitive substrates of P-gp or breast cancer resistance protein (BCRP). This increased monitoring allows for early detection and management of potential drug-drug interactions.

In some embodiments, the Cₘₐₓ of unbound futibatinib in a subject with hepatic impairment is increased compared to a subject with normal hepatic function after administration of about 20 mg of futibatinib once daily. In some embodiments the geometric least square mean (geometric LSM) Cₘₐₓ of unbound futibatinib in a subject with normal hepatic function is about 2.06 ng/mL. The ranges for the geometric LSM Cₘₐₓ of unbound futibatinib in a subject with normal hepatic function include about 1.545 ng/mL to about 2.575 ng/mL, about 1.648 ng/mL to about 2.472 ng/mL, about 1.751 ng/mL to about 2.369 ng/mL, about 1.854 ng/mL to about 2.266 ng/mL, about 1.957 ng/mL to about 2.163 ng/mL, and any value within any of these ranges. Individual values include about 1.545 ng/mL, about 1.648 ng/mL, about 1.751 ng/mL, about 1.854 ng/mL, about 1.957 ng/mL, about 2.06 ng/mL, about 2.163 ng/mL, about 2.266 ng/mL, about 2.369 ng/mL, about 2.472 ng/mL, about 2.575 ng/mL, and any range between any two of these values.

In some embodiments, the Cₘₐₓ of unbound futibatinib in a subject with hepatic impairment is increased compared to a subject with normal hepatic function after administration of about 20 mg of futibatinib once daily. In some embodiments the mean Cₘₐₓ of unbound futibatinib in a subject with normal hepatic function is about 2.06 ng/mL. The ranges for the mean Cₘₐₓ of unbound futibatinib in a subject with normal hepatic function include about 1.545 ng/mL to about 2.575 ng/mL, about 1.648 ng/mL to about 2.472 ng/mL, about 1.751 ng/mL to about 2.369 ng/mL, about 1.854 ng/mL to about 2.266 ng/mL, about 1.957 ng/mL to about 2.163 ng/mL, and any value within any of these ranges. Individual values include about 1.545 ng/mL, about 1.648 ng/mL, about 1.751 ng/mL, about 1.854 ng/mL, about 1.957 ng/mL, about 2.06 ng/mL, about 2.163 ng/mL, about 2.266 ng/mL, about 2.369 ng/mL, about 2.472 ng/mL, about 2.575 ng/mL, and any range between any two of these values.

In some embodiments, the geometric LSM Cₘₐₓ of unbound futibatinib in a subject with hepatic impairment is increased by about 3.2-fold compared to a subject with normal hepatic function. The ranges for the fold increase in geometric LSM Cₘₐₓ include about 2.4-fold to about 4.0-fold, about 2.56-fold to about 3.84-fold, about 2.72-fold to about 3.68-fold, about 2.88-fold to about 3.52-fold, about 3.04-fold to about 3.36-fold, and any value within any of these ranges. Individual values include about 2.4-fold, about 2.56-fold, about 2.72-fold, about 2.88-fold, about 3.04-fold, about 3.2-fold, about 3.36-fold, about 3.52-fold, about 3.68-fold, about 3.84-fold, about 4.0-fold, and any range between any two of these values.

In some embodiments, the mean Cₘₐₓ of unbound futibatinib in a subject with hepatic impairment is increased by about 3.2-fold compared to a subject with normal hepatic function. The ranges for the fold increase in the mean Cₘₐₓ include about 2.4-fold to about 4.0-fold, about 2.56-fold to about 3.84-fold, about 2.72-fold to about 3.68-fold, about 2.88-fold to about 3.52-fold, about 3.04-fold to about 3.36-fold, and any value within any of these ranges. Individual values include about 2.4-fold, about 2.56-fold, about 2.72-fold, about 2.88-fold, about 3.04-fold, about 3.2-fold, about 3.36-fold, about 3.52-fold, about 3.68-fold, about 3.84-fold, about 4.0-fold, and any range between any two of these values.

In some embodiments, the mean AUC of unbound futibatinib in a subject with hepatic impairment is increased compared to a subject with normal hepatic function after administration of about 20 mg of futibatinib once daily. The geometric LSM AUC of unbound futibatinib in a subject with normal hepatic function is about 7.53. The ranges for the geometric LSM AUC of unbound futibatinib in a subject with normal hepatic function include about 5.6475 to about 9.4125, about 6.0225 to about 9.0375, about 6.3975 to about 8.6625, about 6.7725 to about 8.2875, about 7.1475 to about 7.9125, and any value within any of these ranges. Individual values include about 5.6475, about 6.0225, about 6.3975, about 6.7725, about 7.1475, about 7.53, about 7.9125, about 8.2875, about 8.6625, about 9.0375, about 9.4125, and any range between any two of these values.

In some embodiments, the mean AUC of unbound futibatinib in a subject with hepatic impairment is increased compared to a subject with normal hepatic function after administration of about 20 mg of futibatinib once daily. The mean AUC of unbound futibatinib in a subject with normal hepatic function is about 7.53. The ranges for the mean AUC of unbound futibatinib in a subject with normal hepatic function include about 5.6475 to about 9.4125, about 6.0225 to about 9.0375, about 6.3975 to about 8.6625, about 6.7725 to about 8.2875, about 7.1475 to about 7.9125, and any value within any of these ranges. Individual values include about 5.6475, about 6.0225, about 6.3975, about 6.7725, about 7.1475, about 7.53, about 7.9125, about 8.2875, about 8.6625, about 9.0375, about 9.4125, and any range between any two of these values.

In some embodiments, the mean AUC of unbound futibatinib in a subject with hepatic impairment is increased compared to a subject with normal hepatic function after administration of about 20 mg of futibatinib once daily. The geometric LSM AUC of unbound futibatinib in a subject with normal hepatic function is about 7.53 ng*hr/mL. The ranges for the geometric LSM AUC of unbound futibatinib in a subject with normal hepatic function include about 3.765 ng*hr/mL to about 11.295 ng*hr/mL, about 4.518 ng*hr/mL to about 10.542 ng*hr/mL, about 5.271 ng*hr/mL to about 9.789 ng*hr/mL, about 6.024 ng*hr/mL to about 9.036 ng*hr/mL, about 6.777 ng*hr/mL to about 8.283 ng*hr/mL, and any value within any of these ranges. Individual values include about 3.765 ng*hr/mL, about 4.518 ng*hr/mL, about 5.271 ng*hr/mL, about 6.024 ng*hr/mL, about 6.777 ng*hr/mL, about 7.53 ng*hr/mL, about 8.283 ng*hr/mL, about 9.036 ng*hr/mL, about 9.789 ng*hr/mL, about 10.542 ng*hr/mL, about 11.295 ng*hr/mL, and any range between any two of these values.

In some embodiments, the mean AUC of unbound futibatinib in a subject with hepatic impairment is increased compared to a subject with normal hepatic function after administration of about 20 mg of futibatinib once daily. The mean AUC of unbound futibatinib in a subject with normal hepatic function is about 7.53 ng*hr/mL. The ranges for the mean AUC of unbound futibatinib in a subject with normal hepatic function include about 3.765 ng*hr/mL to about 11.295 ng*hr/mL, about 4.518 ng*hr/mL to about 10.542 ng*hr/mL, about 5.271 ng*hr/mL to about 9.789 ng*hr/mL, about 6.024 ng*hr/mL to about 9.036 ng*hr/mL, about 6.777 ng*hr/mL to about 8.283 ng*hr/mL, and any value within any of these ranges. Individual values include about 3.765 ng*hr/mL, about 4.518 ng*hr/mL, about 5.271 ng*hr/mL, about 6.024 ng*hr/mL, about 6.777 ng*hr/mL, about 7.53 ng*hr/mL, about 8.283 ng*hr/mL, about 9.036 ng*hr/mL, about 9.789 ng*hr/mL, about 10.542 ng*hr/mL, about 11.295 ng*hr/mL, and any range between any two of these values.

In some embodiments, the geometric LSM AUC of unbound futibatinib in a subject with hepatic impairment is increased by about 2.4-fold compared to a subject with normal hepatic function. The ranges for the fold increase in geometric LSM AUC include about 1.8-fold to about 3.0-fold, about 1.92-fold to about 2.88-fold, about 2.04-fold to about 2.76-fold, about 2.16-fold to about 2.64-fold, about 2.28-fold to about 2.52-fold, and any value within any of these ranges. Individual values include about 1.8-fold, about 1.92-fold, about 2.04-fold, about 2.16-fold, about 2.28-fold, about 2.4-fold, about 2.52-fold, about 2.64-fold, about 2.76-fold, about 2.88-fold, about 3.0-fold, and any range between any two of these values.

In some embodiments, the mean AUC of unbound futibatinib in a subject with hepatic impairment is increased by about 2.4-fold compared to a subject with normal hepatic function. The ranges for the fold increase in the mean AUC include about 1.8-fold to about 3.0-fold, about 1.92-fold to about 2.88-fold, about 2.04-fold to about 2.76-fold, about 2.16-fold to about 2.64-fold, about 2.28-fold to about 2.52-fold, and any value within any of these ranges. Individual values include about 1.8-fold, about 1.92-fold, about 2.04-fold, about 2.16-fold, about 2.28-fold, about 2.4-fold, about 2.52-fold, about 2.64-fold, about 2.76-fold, about 2.88-fold, about 3.0-fold, and any range between any two of these values.

In some embodiments, both the mean Cₘₐₓ and mean AUC of unbound futibatinib in a subject with hepatic impairment are increased compared to a subject with normal hepatic function. The simultaneous increase in both Cₘₐₓ and AUC indicates altered pharmacokinetics of futibatinib in subjects with hepatic impairment.

In some embodiments, both the mean Cₘₐₓ and mean AUC of unbound futibatinib in a subject with hepatic impairment are increased compared to a subject with normal hepatic function. The simultaneous increase in both Cₘₐₓ and AUC indicates altered pharmacokinetics of futibatinib in subjects with hepatic impairment.

In some embodiments, the geometric LSM Cₘₐₓ of unbound futibatinib in a subject with mild hepatic impairment is increased by about 1.7-fold compared to a subject with normal hepatic function. The ranges for the fold increase in geometric LSM Cₘₐₓ for mild hepatic impairment include about 1.275-fold to about 2.125-fold, about 1.36-fold to about 2.04-fold, about 1.445-fold to about 1.955-fold, about 1.53-fold to about 1.87-fold, about 1.615-fold to about 1.785-fold, and any value within any of these ranges. Individual values include about 1.275-fold, about 1.36-fold, about 1.445-fold, about 1.53-fold, about 1.615-fold, about 1.7-fold, about 1.785-fold, about 1.87-fold, about 1.955-fold, about 2.04-fold, about 2.125-fold, and any range between any two of these values.

In some embodiments, the mean Cₘₐₓ of unbound futibatinib in a subject with mild hepatic impairment is increased by about 1.7-fold compared to a subject with normal hepatic function. The ranges for the fold increase in mean Cₘₐₓ for mild hepatic impairment include about 1.275-fold to about 2.125-fold, about 1.36-fold to about 2.04-fold, about 1.445-fold to about 1.955-fold, about 1.53-fold to about 1.87-fold, about 1.615-fold to about 1.785-fold, and any value within any of these ranges. Individual values include about 1.275-fold, about 1.36-fold, about 1.445-fold, about 1.53-fold, about 1.615-fold, about 1.7-fold, about 1.785-fold, about 1.87-fold, about 1.955-fold, about 2.04-fold, about 2.125-fold, and any range between any two of these values.

In some embodiments, the geometric LSM AUC of unbound futibatinib in a subject with mild hepatic impairment is increased by about 1.7-fold compared to a subject with normal hepatic function. The ranges for the fold increase in geometric LSM AUC for mild hepatic impairment include about 1.275-fold to about 2.125-fold, about 1.36-fold to about 2.04-fold, about 1.445-fold to about 1.955-fold, about 1.53-fold to about 1.87-fold, about 1.615-fold to about 1.785-fold, and any value within any of these ranges. Individual values include about 1.275-fold, about 1.36-fold, about 1.445-fold, about 1.53-fold, about 1.615-fold, about 1.7-fold, about 1.785-fold, about 1.87-fold, about 1.955-fold, about 2.04-fold, about 2.125-fold, and any range between any two of these values.

In some embodiments, the mean AUC of unbound futibatinib in a subject with mild hepatic impairment is increased by about 1.7-fold compared to a subject with normal hepatic function. The ranges for the fold increase in the mean AUC for mild hepatic impairment include about 1.275-fold to about 2.125-fold, about 1.36-fold to about 2.04-fold, about 1.445-fold to about 1.955-fold, about 1.53-fold to about 1.87-fold, about 1.615-fold to about 1.785-fold, and any value within any of these ranges. Individual values include about 1.275-fold, about 1.36-fold, about 1.445-fold, about 1.53-fold, about 1.615-fold, about 1.7-fold, about 1.785-fold, about 1.87-fold, about 1.955-fold, about 2.04-fold, about 2.125-fold, and any range between any two of these values.

Further aspects of this invention are described in the US label highlights and full prescribing information for Lytgobi® (futibatinib) revised Apr. 17, 2024; TAS-120-108 clinical study report (Celerion Clinical Study Report No. CA 31246); and 1.11.3 Clinical Information Amendment” dated 27 Sep. 2023 for NDA 214801, each of which is herein incorporated by reference in its entirety.

### EXAMPLES

### Example 1: Pharmacokinetic Study of Futibatinib in Subjects with Varying Degrees of Hepatic Function

Summary: A single-dose pharmacokinetic study was conducted to evaluate the effect of hepatic impairment on the pharmacokinetics of futibatinib. The study included subjects with normal hepatic function and subjects with chronic, stable hepatic impairment with features of cirrhosis due to any etiology. Hepatically impaired subjects were classified by both NCI-ODWG criteria and Child-Pugh score.

Methods: Subjects were administered a single oral dose of 20 mg futibatinib. PK sampling was taken predose and up to 72 hours after futibatinib dosing. Blood sampling was also conducted for assessment of futibatinib protein binding.

The appropriate noncompartmental PK parameters were calculated from the plasma futibatinib concentration-time data using Phoenix® WinNonlin® Version 8.3.4. Actual sample times were used in the calculations of the PK parameters. The calculation of the actual time was in respect to the dose administration time of futibatinib on Day 1. All PK parameters included in the protocol are listed in Table 3, and are defined as appropriate for study design.

TABLE 3
Noncompartmental Pharmacokinetic Parameters Calculated

| | Definition | Method of Determination |
|---|---|---|
| AUC₀₋₂₄ | Area under the concentration-time | Calculated using the Linear |
| AUCᵤ₀₋₂₄* | curve from time 0 to Hour 24 postdose. | Trapezoidal with Linear [symbol] |
| AUC₀₋ₜ | Area under the concentration-time | Calculated using the Linear |
| AUCᵤ₀₋ₜ* | curve from time 0 to the time of the | Trapezoidal with Linear |
| | last observed/measured non-zero | Interpolation Method. |
| AUC_{0-inf} | Area under the concentration-time | AUC0-∞ = AUC0-t + (Clast/kel) |
| AUC_{u0-inf}* | curve from time 0 extrapolated to | where Clast is the last |
| | infinity. | observed/measured |
| AUC_{% extrap} | Percent of AUC0-inf extrapolated. | Calculated as |
| | | (1 − AUC0-last/AUC0-inf)*100 |
| Cₘₐₓ | The maximum observed concentration. | Taken directly from bioanalytical |
| Cₘₐₓᵤ* | | data. |
| Tₘₐₓ | The time to reach Cmax. | Taken from clinical database as |
| | | the difference in the time of |
| | | administration and the time of the |
| | | blood draw which was associated [symbol] |
| Kel | Apparent first order terminal | Calculated from a semi log plot of |
| | elimination rate constant. | the plasma concentration versus |
| | | time curve. The parameter was |
| | | calculated by linear least squares |
| | | regression analysis using the |
| | | maximum number of points in the |
| | | terminal log linear phase (e.g., [symbol] |
| t_{1/2} | Apparent first order terminal | Calculated as ln(2)/Kel |
| | elimination half life. | |
| CL/F | The apparent total plasma (or serum) | Calculated as |
| CLu/F* | clearance after extravascular | Dose/(AUC0-∞) |
| | administration. | OR |
| | | Dose corrected/(AUC0-∞) |
| Vz/F | The apparent volume of distribution | Calculated as |
| Vzu/F* | during the terminal elimination phase | Dose/(AUC0-∞ × Kel) |
| | after extravascular administration. | |
| Fu* | Fraction of unbound (free; not protein- | Calculated as: Average of the 2 |
| | bound) drug in plasma. | ratios of unbound concentration |
| | | relative to total concentration at 2 |

*PK parameters were also expressed in terms of unbound concentrations. Unbound PK parameters were calculated using Fu × parameter for AUCs and Cmax and parameter ÷ Fu for CL/F and Vz/F. [symbol]
indicates data missing or illegible when filed

PK parameters were not calculated for subjects with less than 3 consecutive postdose time points with quantifiable concentrations. Subjects for whom there were insufficient data to calculate the PK parameters were included in the concentration tables and concentration table summary statistics but were excluded from the PK parameter table and the PK parameter summary statistics. Details on summary statistics are presented in Section 9.7.1.1 below.

Concentrations below the limit of quantification (BLQ) were assigned a value of zero if they occurred between time zero (predose) and the first measurable plasma concentration after dosing and missing thereafter, and were treated as such for the purpose of calculating PK parameters and summary statistics.

### Drug Concentration Measurements

All concentration data were included in the calculation of the individual PK parameters, the individual concentration-time plots (based on actual sample times), and in the mean concentration-time plots (based on nominal sample times). However, if there were any significant deviations from nominal sample times, some concentration data may have been excluded from mean concentration-time plots and/or additional concentration-time plots of the mean data may have been provided.

Blood samples for the determination of total futibatinib were collected from each subject before dosing (predose) followed by sampling at 0.33, 0.667, 1, 1.5, 2, 2.5, 3, 4, 6, 8, 10, 12,24 (Day 2), 36 (Day 2), 48 (Day 3), and 72 hours (Day 4) postdose.

Blood for the determination of unbound futibatinib was collected at predose, followed by sampling at 2 and 12 hours postdose. If the calculated Fu at both time points (2 and 12 hours) appeared similar, Fu was calculated based on the average of the 2 ratios of unbound concentration (Cu) relative to total concentration (C) at 2 and 12 hours postdose for each subject as follows: Fu=(Cu_{2 hrs}/C_{2 hrs}+Cu_{12 hrs}/C_{12 hrs})÷2. If an unbound futibatinib postdose sample (2 and 12 hours) was BLQ or not available, then the sample was treated as missing, and Fu was calculated as follows:
Where both concentrations were available at 2 and 12 hours, then the average Fu across both time points was calculated.
Where a quantifiable unbound futibatinib concentration was available for 1 out of the 2 time points, Fu was calculated based on that single time point.
If unbound futibatinib concentration was not available for both 2 and 12 hours, Fu was not calculated.

### Analytical Procedures

Samples from all subjects were assayed even if the subjects did not complete the study. Samples were analyzed for plasma futibatinib and plasma unbound futibatinib using validated bioanalytical methods.

Plasma total and unbound concentrations of futibatinib were determined using high performance liquid chromatography-tandem mass spectrometry (HPLC-MS/MS) methods validated with respect to accuracy, precision, linearity, sensitivity, and specificity. The analytical range (lower limit of quantitation [LLOQ]—upper limit of quantitation [ULOQ]) for plasma futibatinib and unbound plasma futibatinib were 0.500-250 ng/mL and 0.050-25.0 ng/mL, respectively.

This trial was performed in accordance with the “ICH E6 Guideline: Good Clinical Practice: Consolidated Guidance” and applicable regulatory requirements. Data were handled and processed according to the principles of GCP.

### Data Summarization and Presentation

All plasma futibatinib PK concentrations and/or PK parameters descriptive statistics were generated using SAS® Version 9.4.

The plasma concentrations of futibatinib were listed and summarized by cohorts and time points for all subjects in the PK Population. Plasma concentrations of futibatinib were presented with the same level of precision as received from the bioanalytical laboratory. Summary statistics, including sample size (n), arithmetic mean (Mean), standard deviation (SD), coefficient of variation (CV %), standard error of the mean (SEM), minimum, median, and maximum were calculated for all nominal concentration time points. Excluded subjects were included in the concentration listings, but were excluded from the summary and inferential statistics and noted as such in the tables. All BLQ values were presented as “BLQ” in the concentration listings and footnoted accordingly.

Mean and individual concentration-time profiles were presented on linear and semi-log scales. Linear mean plots were presented with and without SD.

Plasma futibatinib PK parameters (total and unbound) were listed and summarized by cohorts for all subjects in the PK Population. Pharmacokinetic parameters were reported to 3 significant FIGURES for individual parameters, with the exception of Tmax, which was presented with 2 decimal places. Summary statistics (n, arithmetic mean, SD, CV %, SEM, minimum, median, maximum, geometric mean (Geom Mean) and geometric CV % (Geom CV %) were calculated for plasma futibatinib PK parameters. Subjects excluded from the summary and inferential statistics were listed in the PK parameter tables and noted as such.

### Statistical Analysis of PK Parameters

An analyses of variance (ANOVA) was performed on the natural log (ln)-transformed futibatinib (total and unbound) AUC0-24, AUC0-inf, and Cmax using PROC MIXED of SAS®. The ANOVA model included HI status (cohort) as a fixed effect. The geometric least-squares mean (LSM) values reported in the statistical comparisons were the exponentiated LSMs from the ANOVA. The geometric ratio of the LSMs was calculated from the exponentiated difference between the cohort LSMs from the ANOVA analysis. The geometric ratios were expressed as a percentage relative to the healthy-control cohort. The 90% confidence intervals (CIs) for the ratios were derived by the exponentiation of the CIs obtained for the difference between the cohort LSMs. The CIs were expressed as a percentage relative to the healthy-control cohort. Note that due to expected differences in the variability of PK parameter values between cohorts, the residual variance was estimated separately for each cohort with use of a repeated statement.

The analysis was performed by comparing each HI cohort to the associated matching HI healthy-control cohort and by comparing each HI cohort to the overall healthy-control cohort.

### Results:

### Listing of Total Plasma Futibatinib Pharmacokinetic Parameters Following a Single Oral Dose of 20 mg Futibatinib

Table 4 shows listing of each subject with estimated select individual PK parameters using total futibatinib concentration.

TABLE 4
Total Plasma Futibatinib Pharmacokinetic Parameters Following a
Single Oral Dose of 20 mg Futibatinib in Mild and Moderate
Hepatic Impairment (Child-Pugh A, B and C)

| | | Total | | | | | | | |
|---|---|---|---|---|---|---|---|---|---|
| | | Bilirubin | | | | | | | |
| | | <1.5 × | Cₘₐₓ | | | AUCₗₐₛₜ | AUC₂₄ | AUC_{inf} | AUC_{inf} |
| Subject | Child- | ULN | (ng/ | Tₘₐₓ | Tₗₐₛₜ | (ng*h/ | (ng*h/ | (ng*h/ | (extrap) |
| ID | Pugh | Day −1 | mL) | (h) | (h) | mL) | mL) | mL) | (%) |
| 001005 | HC | yes | 136 | 2.00 | 12 | 396 | 399 | 399 | 0.832 |
| 001006 | HC | yes | 61.0 | 1.00 | 12 | 132 | 134 | 134 | 2.00 |
| 001008 | HC | yes | 55.1 | 1.50 | 12 | 143 | 145 | 145 | 1.13 |
| 001011 | HC | yes | 103 | 1.00 | 12 | 373 | 379 | 379 | 1.66 |
| 001019 | HC | yes | 127 | 4.00 | 24 | 880 | 880 | 884 | 0.423 |
| 001020 | HC | yes | 175 | 3.00 | 24 | 1440 | 1440 | 1450 | 0.889 |
| 001027 | HC | yes | 247 | 2.50 | 24 | 1320 | 1320 | 1320 | 0.316 |
| 001028 | HC | yes | 58.6 | 6.00 | 12 | 287 | 303 | 303 | 5.06 |
| 002013 | HC | yes | 85.1 | 2.00 | 24 | 377 | 377 | 386 | 2.33 |
| 002014 | HC | yes | 97.1 | 1.50 | 24 | 865 | 865 | 872 | 0.773 |
| 002015 | HC | yes | 121 | 0.667 | 8 | 187 | 188 | 188 | 0.416 |
| 002016 | HC | yes | 124 | 2.00 | 12 | 361 | 364 | 364 | 0.651 |
| 002018 | HC | yes | 66.0 | 1.50 | 10 | 178 | 179 | 179 | 0.806 |
| 002019 | HC | yes | 63.8 | 2.00 | 8 | 161 | 162 | 162 | 0.663 |
| 002027 | HC | yes | 185 | 1.00 | 12 | 628 | 637 | 638 | 1.54 |
| 002029 | HC | yes | 247 | 2.00 | 24 | 1280 | 1280 | 1300 | 1.26 |
| 001003 | Mild HI | yes | 77.1 | 1.00 | 12 | 353 | 364 | 364 | 3.09 |
| 001004 | Mild HI | yes | 174 | 1.00 | 24 | 707 | 707 | 710 | 0.422 |
| 001010 | Mild HI | yes | 72.1 | 2.50 | 8 | 173 | 176 | 176 | 1.56 |
| 002001 | Mild HI | yes | 215 | 1.00 | 10 | 362 | 364 | 364 | 0.292 |
| 002002 | Mild HI | yes | 122 | 1.50 | 10 | 317 | 319 | 319 | 0.491 |
| 002006 | Mild HI | yes | 124 | 1.00 | 12 | 566 | 592 | 593 | 4.48 |
| 001001 | Mild HI | yes | 165 | 1.00 | 12 | 364 | 366 | 366 | 0.547 |
| 002012 | Mild HI | yes | 101 | 1.00 | 12 | 230 | 231 | 231 | 0.736 |
| 001013 | Moderate HI | yes | 95.0 | 2.50 | 12 | 356 | 364 | 364 | 2.34 |
| 001015 | Moderate HI | yes | 47.1 | 1.00 | 6 | 66.2 | 67.3 | 67.3 | 1.60 |
| 002009 | Moderate HI | yes | 159 | 1.50 | 24 | 617 | 617 | 621 | 0.72 |
| 002011 | Moderate HI | yes | 134 | 2.00 | 24 | 849 | 849 | 853 | 0.484 |
| 001017 | Moderate HI | yes | 193 | 1.00 | 24 | 786 | 786 | 790 | 0.562 |
| 002010 | Moderate HI | yes | 110 | 3.00 | 12 | 501 | 526 | 527 | 4.90 |
| 002021 | Moderate HI | yes | 178 | 1.00 | 24 | 905 | 905 | 928 | 2.51 |
| 001016 | Moderate HI | no | 197 | 0.667 | 24 | 919 | 919 | 923 | 0.414 |
| 002023 | Severe HI | yes | 166 | 2.00 | 24 | 753 | 753 | 758 | 0.649 |
| 001022 | Severe HI | no | 158 | 3.00 | 48 | 1080 | 1040 | 1090 | 0.730 |
| 001023 | Severe HI | no | 148 | 0.667 | 24 | 640 | 640 | 646 | 1.00 |
| 001024 | Severe HI | no | 76.1 | 1.00 | 12 | 332 | 339 | 340 | 2.15 |
| 001025 | Severe HI | no | 110 | 2.50 | 24 | 193 | 193 | 194 | 0.499 |
| 002020 | Severe HI | no | 133 | 1.50 | 12 | 412 | 413 | 413 | 0.318 |

### Statistical Comparisons of Total Plasma Futibatinib Pharmacokinetic Parameters

The summary of statistical comparison of Cₘₐₓ, AUC₂₄, and AUC_{inf} with geometric mean ratio (90% CI) is presented in Table 4 in subjects with mild, moderate, or severe hepatic impairment versus overall healthy-control subjects with normal hepatic function. The magnitude of futibatinib exposure changes in subjects with mild, moderate, or severe hepatic impairment were not considered to be clinically relevant for either of the classification methods.

TABLE 5
Summary of Statistical Comparisons of Total Plasma Futibatinib Pharmacokinetic
Parameters Following a Single Oral Dose of 20 mg Futibatinib by Hepatic Function

| NCI-ODWG | | | | | Child Pugh | | | | |
|---|---|---|---|---|---|---|---|---|---|
| | Geo- | | | Inter- | | Geo- | | | Inter- |
| | metric | GMR | 90% | subject | | metric | GMR | 90% | subject |
| | LSM | (%) | CI | CV % | | LSM | (%) | CI | CV % |
| C_{max,} (ng/mL) | | | | | C_{max,} (ng/mL) | | | | |
| Normal | 109.6 | — | — | 50.37 | Normal | 108.4 | — | — | 53.26 |
| (n = 26) | | | | | (n = 16) | | | | |
| Mild | 147.9 | 134.92 | 105.61- | 27.46 | Mild | 123.0 | 113.52 | 82.50- | 40.45 |
| (n = 6) | | | 172.36 | | (n = 8) | | | 156.21 | |
| Moderate | 136.8 | 124.85 | 85.12- | 42.77 | Moderate | 127.8 | 117.90 | 82.04- | 51.10 |
| (n = 4) | | | 183.11 | | (n = 8) | | | 169.44 | |
| Severe | 121.0 | 110.36 | 87.95- | 13.49 | Severe | 127.6 | 117.78 | 87.42- | 29.89 |
| (n = 2) | | | 138.48 | | (n = 6) | | | 158.68 | |
| AUC_{24,} (ng*h/mL) | | | | | AUC_{24,} (ng*h/mL) | | | | |
| Normal | 394.4 | — | — | 92.10 | Normal | 415.3 | — | — | 98.51 |
| (n = 25) | | | | | (n = 16) | | | | |
| Mild | 536.5 | 136.03 | 86.86- | 56.55 | Mild | 356.8 | 85.92 | 54.94- | 47.51 |
| (n = 6) | | | 213.05 | | (n = 8) | | | 134.38 | |
| Moderate | 674.7 | 171.08 | 103.69- | 53.54 | Moderate | 507.5 | 122.20 | 64.51- | 107.49 |
| (n = 4) | | | 282.26 | | (n = 8) | | | 231.48 | |
| Severe | 282.8 | 71.70 | 35.67- | 57.76 | Severe | 488.5 | 117.62 | 67.44- | 66.87 |
| (n = 2) | | | 144.12 | | (n = 6) | | | 205.13 | |
| AUC_{inf,} (ng*h/mL) | | | | | AUC_{inf,} (ng*h/mL) | | | | |
| Normal | 395.7 | — | — | 92.47 | Normal | 417.0 | — | — | 98.96 |
| (n = 26) | | | | | (n = 16) | | | | |
| Mild | 540.2 | 136.52 | 86.80- | 57.37 | Mild | 357.2 | 85.66 | 54.70- | 47.65 |
| (n = 6) | | | 214.71 | | (n = 8) | | | 134.15 | |
| Moderate | 685.7 | 173.3 | 103.85- | 55.36 | Moderate | 510.8 | 122.20 | 64.50- | 108.17 |
| (n = 4) | | | 289.18 | | (n = 8) | | | 232.63 | |
| Severe | 283.5 | 71.65 | 35.77- | 57.33 | Severe | 494.4 | 118.57 | 67.46- | 68.57 |
| (n = 2) | | | 143.5 | | (n = 6) | | | 208.38 | |

Parameters were In-transformed prior to analysis.
Geometric least-squares means (LSMs) are calculated by exponentiating the LSMs derived from the ANOVA.
Geometric Mean Ratio (GMR) = 100 × (test/reference) Cohort inter-subject CV % was calculated as 100 × square root(exp[MSE]-1), where MSE = Cohort specific residual variance from ANOVA.

### Unbound Plasma Futibatinib Pharmacokinetic Parameters

Table 6 is a summary of the unbound plasma futibatinib pharmacokinetic parameters following a single oral dose of 20 mg futibatinib.

TABLE 6
Unbound Plasma Futibatinib Pharmacokinetic Parameters Following a
Single Oral Dose of 20 mg Futibatinib

| | NCI-ODWG | | | | Child Pugh | | | |
|---|---|---|---|---|---|---|---|---|
| | Normal | Mild | Moderate | Severe | Normal | Mild | Moderate | Severe |
| Parameter | (n = 26) | (n = 6) | (n = 4) | (n = 2) | (n = 16) | (n = 7) | (n = 8) | (n = 6) |
| Fu Geo Mean | 0.185 | 0.0236 | 0.0359 | 0.0293 | 0.0181 | 0.0179 | 0.0231 | 0.0354 |
| Geo CV % | 20.9 | 23.9 | 30.5 | 5.5 | 17.9 | 22.8 | 18.5 | 18.9 |
| C_{max,u} Geo Mean | 2.06 | 3.49 | 4.91 | 3.54 | 1.96 | 2.37 | 2.95 | 4.51 |
| (ng/mL) GeoCV % | 44.4 | 35.2 | 27.8 | 8.0 | 50.8 | 28.1 | 43.7 | 28.9 |
| AUC_{24,u} Geo Mean | 7.53 | 12.7 | 24.2 | 8.28 | 7.51 | 7.05 | 11.7 | 17.3 |
| (ng*hr/mL) Geo CV % | 84.4 | 76.0 | 43.9 | 51.1 | 94.4 | 41.9 | 94.3 | 77.7 |
| AUC_{inf,u} Geo Mean | 7.56 | 12.8 | 24.6 | 8.30 | 7.54 | 7.06 | 11.8 | 17.5 |
| (ng*hr/mL) Geo CV % | 84.8 | 76.9 | 46.4 | 50.7 | 94.8 | 42.0 | 95.0 | 79.6 |
| CLᵤ/F Geo Mean | 2650 | 1570 | 812 | 2410 | 2650 | 283.0 | 1700 | 1140 |
| (L/hr) Geo CV % | 84.8 | 76.9 | 46.4 | 50.7 | 94.8 | 42.0 | 95.0 | 79.6 |
| Vz,ᵤ/F Geo Mean | 8040 | 6320 | 4420 | 4490 | 8110 | 7630 | 6880 | 4480 |
| (L) Geo CV % | 49.6 | 32.7 | 19.4 | 46.6 | 60.9 | 21.6 | 38.5 | 24.8 |

Note:
Fu and all associated unbound parameters are not available for Subject ID 001-010 because the plasma unbound concentration at 2 hours and unbound (buffer) concentration at 12 hours were below the limit of quantitation.

### Summary of Statistical Comparisons of Unbound Plasma Futibatinib Pharmacokinetic Parameters Following a Single Oral Dose of 20 mg Futibatinib by Hepatic Function

The results from Tables 6 and 7 and related data demonstrate the impact of hepatic impairment on the pharmacokinetics of unbound futibatinib. When classified using NCI-ODWG criteria, subjects with mild, moderate, and severe hepatic impairment showed increased unbound futibatinib exposure compared to those with normal hepatic function. The geometric mean ratios (GMRs) for C_{max,u} (Cₘₐₓ, unbound futibatinib) were 169.55a, 238.45, and 171.94 for mild, moderate, and severe impairment, respectively. Similarly, AUC₂₄,u and AUC_{inf,u} showed increased exposure, with the most pronounced effect in the moderate impairment group (GMIRs of 321.71% and 325.84%, respectively). When using the Child-Pugh classification, a trend of increasing exposure with increasing hepatic impairment severity was observed. The GMRs for C_{max,u} were 121.11, 150.39%, and 230.49% for mild, moderate, and severe impairment, respectively. AUC₂₄, and AUC_{inf,u} followed a similar pattern, with the highest exposure increase seen in the severe impairment group (GMIRs of 230.18% and 232.03%, respectively). Interestingly, the two classification methods yielded somewhat different results, particularly for the severe impairment group.

TABLE 7
Summary of Statistical Comparisons of Unbound Plasma Futibatinib Pharmacokinetic
Parameters Following a Single Oral Dose of 20 mg Futibatinib by Hepatic Function

| | NCI-ODWG | | | | Child Pugh | | | | |
|---|---|---|---|---|---|---|---|---|---|
| | Geo- | | | Inter- | | Geo- | | | Inter- |
| | metric | GMR | 90% | subject | | metric | GMR | 90% | subject |
| | LSM | (%) | CI | CV% | | LSM | (%) | CI | CV% |
| Cmax, u (ng/ml) | | | | | Cmax, u (ng/ml) | | | | |
| Normal | 2.06 | — | — | 44.42 | Normal | 7.96 | — | — | 50.83 |
| (n = 25) | | | | | (n = 16) | | | | |
| Mild | 3.49 | 169.55 | 128.48- | 35.18 | Mild | 2.37 | 121.11 | 92.16- | 28.09 |
| (n = 6) | | | 223.75 | | (n = 7) | | | 159.16 | |
| Moderate | 4.91 | 238.45 | 181.34- | 27.82 | Moderate | 2.95 | 150.39 | 108.46- | 43.72 |
| (n = 4) | | | 313.56 | | (n = 8) | | | 208.52 | |
| Severe | 3.54 | 171.94 | 144.51- | 7.96 | Severe | 4.51 | 230.49 | 172.97- | 28.88 |
| (n = 2) | | | 204.59 | | (n = 6) | | | 307.15 | |
| AUC_{24,u} (ng*h/mL) | | | | | AUC_{24,u} (ng*h/mL) | | | | |
| Normal | 7.53 | — | — | 84.43 | Normal | 7.51 | — | — | 94.40 |
| (n = 25) | | | | | (n = 16) | | | | |
| Mild | 12.68 | 168.31 | 99.01- | 76.01 | Mild | 7.05 | 93.95 | 61.02- | 41.89 |
| (n = 6) | | | 286.13 | | (n = 7) | | | 144.67 | |
| Moderate | 24.23 | 321.71 | 207.97- | 43.91 | Moderate | 11.70 | 155.87 | 86.12- | 94.33 |
| (n = 4) | | | 497.65 | | (n = 8) | | | 282.12 | |
| Severe | 8.28 | 109.98 | 58.37- | 51.11 | Severe | 17.28 | 230.18 | 127.10- | 77.71 |
| (n = 2) | | | 207.23 | | (n = 6) | | | 416.86 | |
| AUC_{inf,u} (ng*h/mL) | | | | | AUC_{inf,u} (ng*h/mL) | | | | |
| Normal | 7.56 | — | — | 84.79 | Normal | 7.54 | — | — | 94.83 |
| (n = 25) | | | | | (n = 16) | | | | |
| Mild | 12.76 | 168.89 | 98.92- | 76.92 | Mild | 7.06 | 93.68 | 60.75- | 42.03 |
| (n = 6) | | | 288.37 | | (n = 7) | | | 144.45 | |
| Moderate | 24.62 | 325.84 | 207.34- | 46.41 | Moderate | 11.77 | 156.24 | 86.08- | 95.04 |
| (n = 4) | | | 512.06 | | (n = 8) | | | 283.61 | |
| Severe | 8.30 | 109.9 | 58.53- | 50.70 | Severe | 17.49 | 232.03 | 127.06- | 79.60 |
| (n = 2) | | | 206.34 | | (n = 6) | | | 423.70 | |

Geometric least-squares means (LSMs) are calculated by exponentiating the LSMs derived from the ANOVA.
Geometric Mean Ratio (GMR) = 100 × (test/reference) Cohort inter-subject CV% was calculated as 100 × square root(exp[MSE]-1), where MSE = Cohort specific residual variance from ANOVA.

The results from Table 7 demonstrate changes in the pharmacokinetics of unbound futibatinib in subjects with varying degrees of hepatic impairment compared to those with normal hepatic function.

C_{max,u}: Using NCI-ODWG criteria, the geometric mean ratio (GMR) for C_{max,u} increased by 69.55% in subjects with mild hepatic impairment, 138.45% in moderate impairment, and 71.94% in severe impairment compared to subjects with normal hepatic function. Using Child-Pugh classification, increases of 21.11%, 50.39%, and 130.49% were observed for mild, moderate, and severe impairment, respectively.

AUC_{24,u} and AUC_{inf,u}: For AUC_{24,u} using NCI-ODWG criteria, increases of 68.31%, 221.71%, and 9.98% were observed for mild, moderate, and severe hepatic impairment, respectively. Using Child-Pugh classification, changes of −6.05%, 55.87%, and 130.18% were noted for mild, moderate, and severe impairment.

Similar trends were observed for AUC_{inf,u}, with increases of 68.89%, 225.84%, and 9.89% for mild, moderate, and severe impairment using NCI-ODWG criteria, and changes of −6.32%, 56.24%, and 132.03% using Child-Pugh classification.

These results support the claims of increased Cₘₐₓ and AUC in subjects with hepatic impairment compared to those with normal hepatic function. The magnitude of these increases varies depending on the severity of hepatic impairment and the classification system used. The data suggest that futibatinib exposure may be higher in patients with hepatic impairment, which may inform dosing strategies and monitoring protocols for these patient populations.

All publications mentioned herein are incorporated by reference in their entirety. Nothing herein is to be construed as an admission that the disclosure is not entitled to antedate such disclosure by virtue of prior disclosure.

All features disclosed in the specification, including the abstract and drawings, and all the steps in any method or process disclosed, may be combined in any combination, except combinations where at least some of such features and/or steps are mutually exclusive. Each feature disclosed in the specification, including abstract and drawings, can be replaced by alternative features serving the same, equivalent or similar purpose, unless expressly stated otherwise. Thus, unless expressly stated otherwise, each feature disclosed is one example only of a generic series of equivalent or similar features. Various modifications of the disclosure, in addition to those described herein, will be apparent to those skilled in the art from the foregoing description. Such modifications are also intended to fall within the scope of the appended claims.

1. A method of treating cholangiocarcinoma in a subject with cirrhosis and total bilirubin >1.5 times the upper limit of normal up to 3 times the upper limit of normal, comprising orally administering to the subject 20 mg of futibatinib once a day.

2. The method of claim 1, wherein the subject has previously treated, unresectable, locally advanced or metastatic intrahepatic cholangiocarcinoma harboring fibroblast growth factor receptor 2 (FGFR2) gene fusions or other rearrangements.

3. The method of claim 1, wherein a mean Cₘₐₓ of unbound futibatinib in the subject after administration of the futibatinib to the subject is increased compared to a mean Cₘₐₓ of unbound futibatinib of a subject with normal hepatic function after administration of 20 mg of futibatinib once daily.

4. The method of claim 1, wherein a mean AUC of unbound futibatinib in the subject after administration of the futibatinib to the subject is increased compared to a mean AUC of unbound futibatinib of a subject with normal hepatic function after administration of 20 mg of futibatinib once daily.

5. The method of claim 1, wherein the subject is monitored for an increased risk of one or more adverse reactions compared to risk of one or more adverse reactions in a subject with normal hepatic function, wherein the increased risk of one or more adverse reactions is attributable to higher unbound futibatinib in the plasma of the subject.

6. The method of claim 1, wherein a mean Cₘₐₓ and a mean AUC of unbound futibatinib in the subject after administration of the futibatinib to the subject is increased compared to a mean Cₘₐₓ and mean AUC of unbound futibatinib in a subject with normal hepatic function after administration of 20 mg of futibatinib once daily.

7. The method of claim 3, wherein the mean Cₘₐₓ of the unbound futibatinib in the subject after administration of the futibatinib to the subject is increased by 3.2-fold compared to the mean Cₘₐₓ of unbound futibatinib in a subject with normal hepatic function after administration of 20 mg of futibatinib once daily.

8. The method of claim 4, wherein the mean AUC of the unbound futibatinib in the subject after administration of the futibatinib to the subject is increased by 2.4-fold compared with the mean AUC of unbound futibatinib in a subject with normal hepatic function after administration of 20 mg of futibatinib once daily.

9. The method of claim 7, wherein the mean Cₘₐₓ of unbound futibatinib in a subject with normal hepatic function after administration of 20 mg of futibatinib once daily. is about 2.06 ng/mL

10. The method of claim 8, wherein the mean AUC of unbound futibatinib in a subject with normal hepatic function after administration of 20 mg of futibatinib once daily. is about 7.53 ng*hr/mL.

11. The method of claim 5, wherein the adverse reaction is selected from the group consisting of a skin and subcutaneous tissue disorder, a gastrointestinal disorder, fatigue, a metabolism and nutrition disorder, a musculoskeletal and connective tissue disorder, an eye disorder, a nervous system disorder, an infection, weight decrease and any combination thereof.

12. The method of claim 5, wherein the adverse reaction is selected from the group consisting of nail toxicity, nail disorder, nail discoloration, nail dystrophy, nail hypertrophy, nail infection, nail pigmentation, onychalgia, onychoclasis, onycholysis, onychomadesis, onychomycosis, paronychia, alopecia, dry skin, palmar-plantar erythrodysesthesia syndrome, constipation, diarrhea, colitis, gastroenteritis, dry mouth, stomatitis, glossitis, mouth ulceration, mucosal inflammation, pharyngeal inflammation, tongue ulceration, abdominal pain, abdominal discomfort, abdominal pain upper, gastrointestinal pain, hepatic pain, nausea, vomiting, hematemesis, fatigue, asthenia, decreased appetite, musculoskeletal pain, back pain, bone pain, musculoskeletal chest pain, musculoskeletal discomfort, musculoskeletal pain, musculoskeletal stiffness, myalgia, neck pain, non-cardiac chest pain, pain in extremity, spinal pain, arthralgia, arthritis, dry eye, keratitis, lacrimation increased, photokeratitis, punctate keratitis, ulcerative keratitis, dysgeusia, ageusia, taste disorder, urinary tract infection, cystitis, dysuria, weight decrease and any combination thereof.

13. The method of claim 11, wherein the risk of one or more adverse reactions in a subject with normal hepatic function is defined in Table A:
TABLE A

| | All Grades | Grade 3 |
|---|---|---|
| Adverse Reaction | (%)* | (%)* |
| One or more of nail toxicity, nail disorder, nail discoloration, | 47 | 1.9 |
| nail dystrophy, nail hypertrophy, nail infection, nail | | |
| pigmentation, onychalgia, onychoclasis, onychomadesis, | | |
| onychomycosis, or paronychia | | |
| Alopecia | 34 | 0 |
| Dry skin | 29 | 0 |
| Palmar-plantar erythrodysesthesia syndrome | 21 | 4.9 |
| Constipation | 39 | 0 |
| One or more of diarrhea, colitis, or gastroenteritis | 39 | 1 |
| Dry mouth | 35 | 0 |
| One or more of stomatitis, glossitis, mouth ulceration, mucosal | 30 | 6 |
| inflammation, pharyngeal inflammation, or tongue ulceration | | |
| One or more of abdominal pain, abdominal discomfort, | 30 | 2.9 |
| abdominal pain upper, gastrointestinal pain, or hepatic pain | | |
| Nausea | 24 | 1.9 |
| One or more of vomiting or hematemesis | 20 | 1 |
| One or more of fatigue or asthenia | 37 | 8 |
| Decreased appetite | 23 | 2.9 |
| One or more of musculoskeletal pain back pain, bone pain, | 43 | 3.9 |
| musculoskeletal chest pain, musculoskeletal discomfort, | | |
| musculoskeletal pain, musculoskeletal stiffness, myalgia, neck | | |
| pain, non-cardiac chest pain, pain in extremity, or spinal pain | | |
| One or more of arthralgia or arthritis i | 25 | 0 |
| One or more of dry eye, keratitis, lacrimation increased, | 25 | 1 |
| photokeratitis, punctate keratitis, or ulcerative keratitis | | |
| One or more of dysgeusia, ageusia, and taste disorder | 25 | 0 |
| One or more of urinary tract infection, cystitis, or dysuria l | 23 | 2.9 |
| Weight decreased | 18 | 3.9 |

*per NCI CTCAE 4.03

14. The method of claim 5, wherein the adverse reaction is selected from the group consisting of pyrexia, gastrointestinal hemorrhage, ascites, musculoskeletal pain, bile duct obstruction and combinations thereof.

15. The method of claim 14, wherein the risk of one or more adverse reactions in a subject with normal hepatic function is defined in Table B
TABLE B

| Adverse Reaction | Grade 3 (%)* |
|---|---|
| pyrexia | 3.9 |
| gastrointestinal hemorrhage | 3.9 |
| Ascites | 2.9 |
| musculoskeletal pain | 2.9 |
| bile duct obstruction | 2.9 |

*per NCI CTCAE 4.03

16. A method of treating cholangiocarcinoma in a subject with cirrhosis and total bilirubin >1.0 to 1.5 times the upper limit of normal, comprising orally administering to the subject, futibatinib at a dose of 20 mg daily.

17. The method of claim 16, wherein the subject has previously treated, unresectable, locally advanced or metastatic intrahepatic cholangiocarcinoma harboring fibroblast growth factor receptor 2 (FGFR2) gene fusions or other rearrangements.

18. The method of claim 16, wherein the subject is not monitored for an increased risk of one or more adverse reactions compared to a risk of adverse reactions in a subject with normal hepatic function after administration of 20 mg of futibatinib.

19. The method of claim 16, wherein a mean Cₘₐₓ of unbound futibatinib in the subject after administration of the futibatinib is increased compared with a mean Cₘₐₓ of unbound futibatinib in a subject with normal hepatic function after administration of 20 mg of futibatinib.

20. The method of claim 18, wherein a mean AUC of unbound futibatinib in the subject after administration of the futibatinib is increased after administration of the futibatinib, compared with an AUC of unbound futibatinib in a subject with normal hepatic function after administration of 20 mg of futibatinib.

21. The method of claim 16, wherein a mean Cₘₐₓ and a mean AUC of unbound futibatinib in the subject is increased after administration of the futibatinib, compared with a mean Cₘₐₓ and mean AUC of unbound futibatinib in a subject with normal hepatic function after administration of 20 mg of futibatinib.

22. The method of claim 19, wherein the mean Cₘₐₓ of the subject is increased by 1.7-fold compared with the mean Cₘₐₓ of the subject with normal hepatic function.

23. The method of claim 20, wherein the mean AUC of the subject is increased by 1.7-fold compared with the mean AUC of the subject with normal hepatic function.

24. The method of claim 22, wherein the mean Cₘₐₓ of unbound futibatinib of the subject with normal hepatic function is about 2.06 ng/mL.

25. The method of claim 23, wherein the mean AUC of unbound futibatinib of the subject with normal hepatic function is about 7.53 ng*hr/mL.

26. A method of treating previously treated, unresectable, locally advanced or metastatic cholangiocarcinoma harboring fibroblast growth factor receptor 2 gene fusions or other rearrangement in a subject with cirrhosis and total bilirubin >1.5 times the upper limit of normal to 3 times the upper limit of normal, comprising orally administering to the subject 20 mg of futibatinib once a day, wherein the dose is not reduced compared with a standard dose for a subject with normal hepatic function and wherein a mean Cₘₐₓ of unbound futibatinib and mean AUC of unbound futibatinib in the subject after administration of the futibatinib is increased compared with a mean Cₘₐₓ of unbound futibatinib and a mean AUC of unbound futibatinib of a subject with normal hepatic function after administration of 20 mg of futibatinib once daily.

27. The method of claim 26, wherein the subject is monitored for increased incidence or severity of one or more adverse reactions compared with the subject with normal hepatic function.

28. The method of claim 26, wherein the mean Cₘₐₓ in the subject is increased by 3.2-fold compared with the mean Cₘₐₓ in the subject with normal hepatic function.

29. The method of claim 26, wherein the mean AUC in the subject is increased by 2.4-fold compared with the mean AUC in the subject with normal hepatic function.

30. The method of claim 26, wherein the mean Cₘₐₓ of unbound futibatinib of the subject with normal hepatic function is about 2.06 ng/mL.

31. The method of claim 26, wherein the mean AUC of unbound futibatinib of the subject with normal hepatic function is about 7.53 ng*hr/mL.

32. The method of claim 27, wherein the adverse reaction is selected from the group consisting of a skin and subcutaneous tissue disorder, a gastrointestinal disorder, fatigue, a metabolism and nutrition disorder, a musculoskeletal and connective tissue disorder, an eye disorder, a nervous system disorder, an infection, weight decrease and combinations thereof.

33. The method of claim 32, wherein the adverse reaction is selected from the group consisting of nail toxicity, nail disorder, nail discoloration, nail dystrophy, nail hypertrophy, nail infection, nail pigmentation, onychalgia, onychoclasis, onycholysis, onychomadesis, onychomycosis, paronychia, alopecia, dry skin, palmar-plantar erythrodysesthesia syndrome, constipation, diarrhea, colitis, gastroenteritis, dry mouth, stomatitis, glossitis, mouth ulceration, mucosal inflammation, pharyngeal inflammation, tongue ulceration, abdominal pain, abdominal discomfort, abdominal pain upper, gastrointestinal pain, hepatic pain, nausea, vomiting, hematemesis, fatigue, asthenia, decreased appetite, musculoskeletal pain, back pain, bone pain, musculoskeletal chest pain, musculoskeletal discomfort, musculoskeletal pain, musculoskeletal stiffness, myalgia, neck pain, non-cardiac chest pain, pain in extremity, spinal pain, arthralgia, arthritis, dry eye, keratitis, lacrimation increased, photokeratitis, punctate keratitis, ulcerative keratitis, dysgeusia, ageusia, taste disorder, urinary tract infection, cystitis, dysuria, weight decrease and combinations thereof.

34. The method of claim 33, wherein the risk of one or more adverse reactions in a subject with normal hepatic function is defined in Table A:
TABLE A

| | All Grades | Grade 3 |
|---|---|---|
| Adverse Reaction | (%)* | (%)* |
| One or more of nail toxicity, nail disorder, nail discoloration, | 47 | 1.9 |
| nail dystrophy, nail hypertrophy, nail infection, nail | | |
| pigmentation, onychalgia, onychoclasis, onychomadesis, | | |
| onychomycosis, or paronychia | | |
| Alopecia | 34 | 0 |
| Dry skin | 29 | 0 |
| Palmar-plantar erythrodysesthesia syndrome | 21 | 4.9 |
| Constipation | 39 | 0 |
| One or more of diarrhea, colitis, or gastroenteritis | 39 | 1 |
| Dry mouth | 35 | 0 |
| One or more of stomatitis, glossitis, mouth ulceration, mucosal | 30 | 6 |
| inflammation, pharyngeal inflammation, or tongue ulceration | | |
| One or more of abdominal pain, abdominal discomfort, | 30 | 2.9 |
| abdominal pain upper, gastrointestinal pain, or hepatic pain | | |
| Nausea | 24 | 1.9 |
| One or more of vomiting or hematemesis | 20 | 1 |
| One or more of fatigue or asthenia | 37 | 8 |
| Decreased appetite | 23 | 2.9 |
| One or more of musculoskeletal pain back pain, bone pain, | 43 | 3.9 |
| musculoskeletal chest pain, musculoskeletal discomfort, | | |
| musculoskeletal pain, musculoskeletal stiffness, myalgia, neck | | |
| pain, non-cardiac chest pain, pain in extremity, or spinal pain | | |
| One or more of arthralgia or arthritis i | 25 | 0 |
| One or more of dry eye, keratitis, lacrimation increased, | 25 | 1 |
| photokeratitis, punctate keratitis, or ulcerative keratitis | | |
| One or more of dysgeusia, ageusia, and taste disorder | 25 | 0 |
| One or more of urinary tract infection, cystitis, or dysuria l | 23 | 2.9 |
| Weight decreased | 18 | 3.9 |

*per NCI CTCAE 4.03

35. The method of claim 27, wherein the adverse reaction is selected from the group consisting of pyrexia, gastrointestinal hemorrhage, ascites, musculoskeletal pain, bile duct obstruction and combinations thereof.

36. The method of claim 35, wherein the risk of one or more adverse reactions in a subject with normal hepatic function is defined in Table B:
TABLE B

| Adverse Reaction | Grade 3 (%)* |
|---|---|
| pyrexia | 3.9 |
| gastrointestinal hemorrhage | 3.9 |
| Ascites | 2.9 |
| musculoskeletal pain | 2.9 |
| bile duct obstruction | 2.9 |

*per NCI CTCAE 4.03

37. A method of treating cholangiocarcinoma in a subject with moderate to severe hepatic impairment as defined by Child-Pugh B to C and with total bilirubin <1.5 times the upper limit of normal comprising orally administering to the subject 20 mg of futibatinib once a day.

38. The method of claim 37, wherein the subject has previously treated, unresectable, locally advanced or metastatic intrahepatic cholangiocarcinoma harboring fibroblast growth factor receptor 2 (FGFR2) gene fusions or other rearrangements.

39. The method of claim 1, wherein the subject is monitored for hyperphosphatemia and appropriate phosphate lowering therapy is initiated if serum phosphate is ≥5.5 mg/dL.

40. The method of claim 1, wherein the subject is monitored by comprehensive ophthalmological examination including optical coherence tomography (OCT) prior to initiation of therapy, every 2 months for the first 6 months, and every 3 months thereafter.

41. The method of claim 1, wherein the subject does not have a Child-Pugh score designation of A.

42. The method of claim 16, wherein the subject is monitored for hyperphosphatemia and appropriate phosphate lowering therapy is initiated if serum phosphate is ≥5.5 mg/dL.

43. The method of claim 16, wherein the subject is monitored by comprehensive ophthalmological examination including optical coherence tomography (OCT) prior to initiation of therapy, every 2 months for the first 6 months, and every 3 months thereafter.

44. The method of claim 16, wherein the subject does not have a Child-Pugh score designation of A.

45. The method of claim 26, wherein the subject is monitored for hyperphosphatemia and appropriate phosphate lowering therapy is initiated if serum phosphate is ≥5.5 mg/dL.

46. The method of claim 26, wherein the subject is monitored by comprehensive ophthalmological examination including optical coherence tomography (OCT) prior to initiation of therapy, every 2 months for the first 6 months, and every 3 months thereafter.

47. The method of claim 26, wherein the subject does not have a Child-Pugh score designation of A.

48. The method of claim 37, wherein the subject is monitored for hyperphosphatemia and appropriate phosphate lowering therapy is initiated if serum phosphate is ≥5.5 mg/dL.

49. The method of claim 37, wherein the subject is monitored by comprehensive ophthalmological examination including optical coherence tomography (OCT) prior to initiation of therapy, every 2 months for the first 6 months, and every 3 months thereafter.

50. The method of claim 37, wherein the subject does not have a Child-Pugh score designation of A.
